(19) United States
(12) Patent Application Publication
(10) Pub. No.: US 2019/0127747 A1
(43) Pub. Date: May 2, 2019

(54) SEED-SPECIFIC AND ENDOSPERM-PREFERENTIAL PROMOTERS AND USES THEREOF

(71) Applicant: BAYER CROPSCIENCE NV, Diegem (BE)

(72) Inventors: Peter DENOLF, Velzeke (BE); Katrien VAN AUDENHOVE, Gent (BE); John TESKE, Gent (BE)

(21) Appl. No.: 16/092,734

(22) PCT Filed: Apr. 6, 2017
(86) PCT No.: PCT/EP2017/058214

§ 371 Date: Oct. 10, 2018

(30) Foreign Application Priority Data
Apr. 11, 2016 (EP) 16164664.1

(51) Int. Cl. C12N 15/82

(52) U.S. Cl. CPC C12N 15/8234

(57) ABSTRACT

The present invention relates to Brassica sequences comprising early stage seed-specific and endosperm preferential promoter activity. Provided are recombinant genes comprising the early stage seed-specific and endosperm preferential promoter operably linked to a heterologous nucleic acid sequence, and cells, plants and seeds comprising the recombinant gene. The promoters can be used to alter gene expression specifically in the seeds at early developmental stages and preferentially in the endosperm and to alter biotic or abiotic stress tolerance, yield, seed quality or seed properties.

### FIELD OF THE INVENTION

The present invention relates to materials and methods for the expression of a gene of interest specifically in seeds of plants. In particular, the invention provides an expression cassette for regulating seed-specific and endosperm-preferential expression in plants.

### BACKGROUND

Modification of plants to alter and/or improve phenotypic characteristics (such as productivity or quality) requires the overexpression or down-regulation of endogenous genes or the expression of heterologous genes in plant tissues. Such genetic modification relies on the availability of a means to drive and to control gene expression as required. Indeed, genetic modification relies on the availability and use of suitable promoters which are effective in plants and which regulate gene expression so as to give the desired effect(s) in the transgenic plant.

For numerous applications in plant biotechnology a tissue-specific or a tissue-preferential expression profile is advantageous, since beneficial effects of expression in one tissue may have disadvantages in others.

Seed-preferential or seed-specific promoters are useful for expressing or down-regulating genes specifically in the seeds to get the desired function or effect, such as improving disease resistance, herbicide resistance, modifying seed or grain composition or quality, such as modifying starch quality or quantity, modifying oil quality or quantity, modifying amino-acid or protein composition, improving tolerance to biotic or abiotic stress, increasing yield, or altering metabolic pathways in the seeds.

Examples of seed-preferential or seed-specific promoters include the Tonoplast Intrinsic Protein alpha promoter from Arabidopsis thaliana (US patent application US2009/0241230), the KNAT411 promoter from Arabidopsis thaliana (U.S. Pat. No. 6,342,657), an oleosin promoter, a 2S storage protein promoter or a legumin-like seed storage protein promoter from Linum usitatissimum (U.S. Pat. No. 7,642,346), the acyl carrier protein promoter from Brassica napus (US Pat. Application No. 1994/0129129), the β-amylase promoter of barley (US Pat. Application No. 1997/0793599), and the Ha ds10 G1 promoter of sunflower (U.S. Pat. No. 6,759,570).

Several endosperm-preferential promoters have been identified from cereals, such as rice (Wu et al., 1998, Plant Cell Physiol 39: 885; Russell and Fromm, 1997, Transgenic Res 6:157; Hwang et al., 2002, Plant Cell Rep 9:842; US 2011/0093984, U.S. Pat. No. 8,552,256), maize (U.S. Pat. No. 7,071,378, US 2007/0169226, US 2009/0227013, WO 2005/042745, WO 2010/147825; WO 2012/159891), wheat (Lamacchia et al., 2001, J Exp Bot 52:243; Song et al., 2012, Z Naturforsch C 67:611; WO 2010/129999, WO 2010/118477), and barley (US 2007/0199106, WO 1998/008961). Endosperm-preferential promoters have also been identified in the model plant Arabidopsis (WO2007/110600; Tiwari et al., 2006, Plant Biotech J. 4: 393). Knowledge on endosperm-preferential promoters from exalbuminous crop plants or from oil-accumulating crop plants is limited. Huang et al., 2009, BMC Genomics 10:256 have identified about 1200 genes expressed in the endosperm of Brassica napus during embryo development in Brassica napus. However, this study did not compare expression of the genes in the endosperm to expression in other plant tissues, and focused on very early seed-developmental stages. Further, Huang et al did not identify the sequences of the promoters of the genes accumulating in the endosperm.

There remains thus an interest in isolating novel early stage seed-specific promoters having endosperm-preferential activity. It is thus an objective of the present invention to provide Brassica promoters having early stage seed-specific activity and endosperm-preferential activity. This objective is solved by the present invention as herein further explained.

### SUMMARY OF THE INVENTION

In one aspect, the invention provides an isolated nucleic acid comprising early stage seed-specific and endosperm-preferential promoter activity selected from the group consisting of (a) a nucleic acid comprising a nucleotide sequence of any one of SEQ ID NOs: 2 to 7 or a functional fragment thereof; and (b) a nucleic acid comprising a nucleotide sequence having at least 80% sequence identity to any one of SEQ ID NOs: 2 to 7 or a functional fragment thereof.

A further embodiment provides a recombinant gene comprising the nucleic acid according to the invention operably linked to a heterologous nucleic acid sequence encoding an expression product of interest, and optionally a transcription termination and polyadenylation sequence, preferably a transcription termination and polyadenylation region functional in plant cells. In a further embodiment, said expression product of interest is an RNA capable of modulating the expression of a gene or is a protein.

Yet another embodiment provides a host cell, such as an E. coli cell, an Agrobacterium cell, a yeast cell, an algal cell, or a plant cell, comprising the isolated nucleic acid according to the invention, or the recombinant gene according to the invention.

In a further embodiment, a plant is provided comprising the recombinant gene according to the invention. Yet a further embodiment provides seeds obtainable from the plant according to the invention. In another embodiment, the plants or plant parts according to the invention are seed crop plants or seeds.

Yet another embodiment provides a method of producing a transgenic plant comprising the steps of (a) introducing or providing the recombinant gene according to the invention to a plant cell to create transgenic cells; and (b) regenerating transgenic plants from said transgenic cell.

Further provided is a method of effecting early stage seed-specific and endosperm-preferential expression of a nucleic acid comprising introducing the recombinant gene according to the invention into the genome of a plant, or providing the plant according to the invention. Also provided is a method for altering seed properties of a plant or to produce a commercially relevant product in a plant, said method comprising introducing the recombinant gene according to the invention into the genome of a plant, or providing the plant according to the invention. In another embodiment, said plant is a seed crop plant.

Also provided is the use of the isolated nucleic acid according to the invention to regulate expression of an operably linked nucleic acid in a plant, and the use of the isolated nucleic acid according to the invention, or the recombinant gene according to the invention to alter seed properties of a plant or to produce a commercially relevant product in a plant. In a further embodiment, said plant is a seed crop plant.

Yet another embodiment provides a method of producing food, feed, or an industrial product comprising (a) obtaining the plant or a part thereof, according to the invention; and (b) preparing the food, feed or industrial product from the plant or part thereof. In another embodiment, said food or feed is oil, meal, grain, starch, flour or protein, or said industrial product is biofuel, fiber, industrial chemicals, a pharmaceutical or a nutraceutical.

### BRIEF DESCRIPTION OF THE DRAWINGS

FIG. 1: Expression profile analysis in seeds carrying Penodl 10 BnC::GUS. GUS labelling in the endosperm and seed coat at early stage (A, B) and at late stage (C).

FIG. 2: GUS staining in the embryos carrying Penodl 110 BnC::GUS. A: GUS labelling in whole embryos at different developmental stages: sub-panel A: 10 to 12 DAF, sub-panel B: 15 to 18 DAF, sub-panel C: 20 to 23 DAF; sub-panel D: 23 to 27 DAF; sub-panel E: 27 to 32 DAF; sub-panel F: 35 to 40 DAF. B: GUS labelling in sectioned embryos at late stage.

FIG. 3: Semi quantitative assessment of GUS labelling (μU*mg⁻¹ fresh weight*h) in transgenic lines carrying a Penodl 110 BnC::GUS T-DNA. Seed coats (A) and embryos (B) stained at the following stages: 2: 13 to 15 DAF, 3: 16 to 18 DAF, 3: 19 to 23 DAF, 4: 25 to 29 DAF, 6: 31 to 35 DAF, 7: 37 to 40 DAF.

FIG. 4: Alignment of the amino acid sequence of different Brassica ENODL10 proteins. Amino acid residues conserved in all proteins are indicated by an asterisk, conserved substitutions are indicated by a colon. The lowest homology level between two ENODL10 proteins is about 80%.

FIG. 5: Relative expression levels of different ENODL10 transcripts in different plant tissues. A: ENODL10 BnC; B: ENODL10 BnA; C: ENODL10 Bo; D: ENODL10 Br; E: ENODL10 BjB; F: ENODL10 BjA. Different tissues for A to D: AM33: Apical meristem 33 days after sowing (DAS); BFB42: Big flower buds 42 DAS; CTYL10: Cotyledons 10 DAS; OF52: Open flowers 52 DAS; Pod2: Pods 14-20 DAS; Pod3: Pods 21-25 DAS; Ro2w: Roots 14 DAS; SFB42: Small flower buds 42 DAS; Seed2: Seeds 14-20 days after flowering (DAF); Seed3: Seeds 21-25 DAF; Seed4: Seeds 26-30 DAF; SeedS: Seeds 31-35 DAF; Seed6: Seeds 42 DAF; Seed7: Seeds 49 DAF; St2w: Stem 14 DAS; St5w: Stem 33 DAS; YL33: Young leaf 33 DAS. Different tissues for E and F: AM22: Apical meristem 22 days after sowing (DAS); BFB35: Big flower buds 35 DAS; CTYL8: Cotyledons 8 DAS; OF35: Open flowers 35 DAS; Pod2: Pods 14-20 DAS; Pod3: Pods 21-25 DAS; Pod4: Pods 26-30 DAS; Pod5: Pods 31-35 DAS; Ro2w: Roots 14 DAS; SFB35: Small flower buds 35 DAS; Seed2: Seeds 14-20 days after flowering (DAF); Seed3: Seeds 21-25 DAF; Seed4: Seeds 26-30 DAF; Seed5: Seeds 31-35 DAF; Seed6: Seeds 42 DAF; Seed7: Seeds 49 DAF; St2w: Stem 14 DAS; St3w: Stem 22 DAS; YL22: Young leaf 22 DAS; OL22: Old leaf 22 DAS.

FIG. 6: Relative expression levels of different ENODL10 transcripts in different seed sub-tissues. A: ENODL10 BnC; B: ENODL10 BnA; C: ENODL10 Bo; D: ENODL10 Br. Different sub-tissues: a: Endosperm, 18 days after flowering (DAF); b: Endosperm, 24 DAF; c: embryonic hypocotyl, 18 DAF; d: embryonic hypocotyl, 24 DAF; e: embryonic hypocotyl, 28 DAF; f: embryonic hypocotyl, 32 DAF; g: embryonic hypocotyl, 46 DAF; h: embryonic inner cotyledon, 18 DAF; i: embryonic inner cotyledon, 24 DAF; j: embryonic inner cotyledon, 28 DAF; k: embryonic inner cotyledon, 32 DAF; I: embryonic inner cotyledon, 46 DAF; m: embryonic outer cotyledon, 18 DAF; n: embryonic outer cotyledon(inner part), 24 DAF; o: embryonic outer cotyledon(inner part), 28 DAF; p: embryonic outer cotyledon(inner part), 32 DAF; q: embryonic outer cotyledon(inner part), 46 DAF; r: embryonic outer cotyledon(outer part), 24 DAF; s: embryonic outer cotyledon(outer part), 28 DAF; t: embryonic outer cotyledon(outer part), 32 DAF; u: embryonic outer cotyledon(outer part), 46 DAF.

FIG. 7: Alignment of the nucleotide sequence of the Brassica Penodl 10 promoters from Brassica napus, Brassica juncea, Brassica oleracea and Brassica rapa. The TATA Box is indicated by a frame. The conserved motifs are underlined and numbered. Motif 1 has the sequence of SEQ ID NO: 20, Motif 1 has the sequence of SEQ ID NO: 20, motif 2 has the sequence of SEQ ID NO: 21, motif 3 has the sequence of SEQ ID NO: 22, motif 4 has the sequence of SEQ ID NO: 23, motif 5 has the sequence of SEQ ID NO: 24, motif 6 has the sequence of SEQ ID NO: 25, motif 7 has the sequence of SEQ ID NO: 26, motif 8 has the sequence of SEQ ID NO: 27, motif 9 has the sequence of SEQ ID NO: 28, motif 10 has the sequence of SEQ ID NO: 29, motif 11 has the sequence of SEQ ID NO: 30, motif 12 has the sequence of SEQ ID NO: 31, motif 13 has the sequence of SEQ ID NO: 32, motif 14 has the sequence caagaaa, motif 15 has the sequence of SEQ ID NO: 33, motif 16 has the sequence of SEQ ID NO: 34, motif 17 has the sequence of SEQ ID NO: 35, motif 18 has the sequence of SEQ ID NO: 36, motif 19 has the sequence of SEQ ID NO: 37, motif 20 has the sequence of SEQ ID NO: 38, motif 21 has the sequence of SEQ ID NO: 39, motif 22 has the sequence of SEQ ID NO: 40, motif 23 has the sequence of SEQ ID NO: 41, motif 24 has the sequence of SEQ ID NO: 42 , motif 25 has the sequence of SEQ ID NO: 43, motif 26 has the sequence taagccaa, motif 27 has the sequence of tttrgmgaa.

### DETAILED DESCRIPTION

The present invention is based on the observation that SEQ ID NOs: 2 to 7 have early stage seed-specific promoter activity and endosperm-preferential promoter activity in Brassica.

SEQ ID NOs: 2 to 7 depict the region upstream (i.e. located 5′ upstream of) from the first ATG start codon of the ENODL10 BnC, ENODL10 BnA, ENODL10 Bo, ENODL10 Br, ENODL10 BjB and ENODL10 BjA, respectively.

ENODL10 BnC and ENODL10 BnA are the two copies present in Brassica napus of the orthologous gene to the Arabidopsis thaliana Early Nodulation-Like 10 gene. ENODL10 Bo is the one copy present in Brassica oleracea of the orthologous gene to the Arabidopsis thaliana Early Nodulation-Like 10 gene. ENODL10 Br is the one copy present in Brassica rapa of the orthologous gene to the Arabidopsis thaliana Early Nodulation-Like 10 gene. ENODL10 BjB and ENODL10 BjA are the two copies present in Brassica juncea of the orthologous gene to the Arabidopsis thaliana Early Nodulation-Like 10 gene. The early nodulin-like gene family is a large plant-specific family of genes initially identified as expressed in legume during the early stages of root nodule development. However, expression analyses in Arabidopsis thaliana and Brassica rapa unraveled the diversity of expression pattern of the different ENODL genes (Mashiguchi et al. 2009 Biosci. Biotechnol. Biochem. 73(11):2452-2459, Li et al. 2013 Mol Genet Genomics 288:1-20). In both studies, the ENODL10 genes were characterized as expressed specifically in flowers but absent from seeds. In contrast Huang et al. identified the Brassica napus orthologs of AtENODL10 as being expressed in the endosperm between 8 and 14 days after flowering, period covering the development of the embryo from the globular-shape stage until entry into the cotyledon stage (Huang et al. 2009 BMC Genomics 10:256).

In one aspect, the invention provides an isolated nucleic acid comprising early stage seed-specific and endosperm-preferential promoter activity selected from the group consisting of (a) a nucleic acid comprising a nucleotide sequence of any one of SEQ ID NOs: 2 to 7 or a functional fragment thereof; and (b) a nucleic acid comprising a nucleotide sequence having at least 80% sequence identity to any one of SEQ ID NOs: 2 to 7, or a functional fragment thereof.

The nucleic acid comprising the early stage seed-specific and endosperm-preferential promoter activity according to the invention may also be comprised in a larger DNA molecule.

“Seed-specific promoter activity” in the context of this invention means the promoter activity is at least 10 times, at least 20 times, or at least 50 times, or at least 100 times, or at least 200 times, or at least 500 times, or even at least 1000 times higher in seeds than in other tissues. In other words, in seed-specific promoter activity, transcription of the nucleic acid operably linked to the promoter of the invention in the seeds is at least 10 times, or at least 20 times, or at least 50 times, or at least 100 times, or at least 200 times, or at least 500 times or even at least 1000 times higher than in other tissues. In other words, the seed-specific promoter drives seed-specific expression of the nucleic acid operably linked to the seed-specific promoter.

“Early stage seed” development in the context of this invention refers to seeds in which the embryo developmental stage ranges from early cotyledon-stage “walking stick” to the curled cotyledon stage. These developmental stages are reached between 14 and 25 days after flowering.

“Seed-specific promoter activity” encompasses “endosperm-preferential promoter activity”.

“Endosperm-preferential promoter activity” in the context of this invention means the promoter activity is at least 2 times, or at least 5 times, or at least 10 times, or at least 20 times or even at least 100 times higher in endosperm tissues than in other seed tissues. In other words, in endosperm-preferential promoter activity, transcription of the nucleic acid operably linked to the promoter of the invention in the endosperm is at least 2 times, or at least 5 times, or at least 10 times, or at least 20 times or even at least 100 times higher than in other seed tissues. In other words, the endosperm-preferential promoter drives endosperm-preferential expression of the nucleic acid operably linked to the endosperm preferential promoter.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

The phrases “DNA”, “DNA sequence,” “nucleic acid sequence,” “nucleic acid molecule” “nucleotide sequence” and “nucleic acid” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

As used herein, “promoter” means a region of DNA sequence that is essential for the initiation of transcription of DNA, resulting in the generation of an RNA molecule that is complementary to the transcribed DNA; this region may also be referred to as a “5′ regulatory region.” Promoters are usually located upstream of the coding sequence to be transcribed and have regions that act as binding sites for RNA polymerase II and other proteins such as transcription factors (trans-acting protein factors that regulate transcription) to initiate transcription of an operably linked gene. Promoters may themselves contain sub-elements (i.e. promoter motifs) such as cis-elements or enhancer domains that regulate the transcription of operably linked genes. The promoters of this invention may be altered to contain “enhancer DNA” to assist in elevating gene expression. As is known in the art, certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (see U.S. Pat. No. 5,593,874), the maize shrunken 1 gene, the light sensitive 1 gene of Solanum tuberosum, the Arabidopsis histon 4 intron and the heat shock protein 70 gene of Petunia hybrida (see U.S. Pat. No. 5,659,122). Thus, as contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed or relative to a promoter driving the expression of a housekeeping gene.

A promoter as used herein may thus include sequences downstream of the transcription start, such as sequences coding the 5′ untranslated region (5′ UTR) of the RNA, introns located downstream of the transcription start, or even sequences encoding the protein. A functional promoter fragment according to the invention may comprise its own 5′UTR comprising the nucleotide sequence of SEQ ID No: 2 from nucleotide 916 to nucleotide 943, or comprising the nucleotide sequence of SEQ ID No: 3 from nucleotide 901 to nucleotide 929, or comprising the nucleotide sequence of SEQ ID No: 4 from nucleotide 914 to nucleotide 943, or comprising the nucleotide sequence of SEQ ID No: 5 from nucleotide 917 to nucleotide 1720, or comprising the nucleotide sequence of SEQ ID No: 6 from nucleotide 934 to nucleotide 978, or comprising the nucleotide sequence of SEQ ID No: 7 from nucleotide 9907 to nucleotide 935. In combination with the above described promoter fragments, a promoter fragment according to the invention may thus comprise the nucleotide sequence of SEQ ID NO: 2 from the nucleotide at position 1 to the nucleotide at position 915, or the nucleotide sequence of SEQ ID NO: 3 from the nucleotide at position 1 to the nucleotide at position 900 or the nucleotide sequence of SEQ ID NO: 4 from the nucleotide at position 1 to the nucleotide at position 913, or the nucleotide sequence of SEQ ID NO: 5 from the nucleotide at position 1 to the nucleotide at position 917, or the nucleotide sequence of SEQ ID NO: 6 from the nucleotide at position 1 to the nucleotide at position 933, or the nucleotide sequence of SEQ ID NO: 7 from the nucleotide at position 1 to the nucleotide at position 906. Alternatively, 5′UTR fragments from other genes may be used.

Promoter activity for a functional promoter fragment in seeds may be determined by those skilled in the art, for example using analysis of RNA accumulation produced from the nucleic acid which is operably linked to the promoter as described herein, whereby the nucleic acid which is operably linked to the promoter can be the nucleic acid which is naturally linked to the promoter, i.e. the endogenous gene of which expression is driven by the promoter.

The early stage seed-specific expression capacity and the endosperm-preferential expression capacity of the identified or generated fragments of the promoters of the invention can be conveniently tested by determining levels of the transcript of which expression is naturally driven by the promoter of the invention, i.e. endogenous transcript levels, such as, for example, using the methods as described herein in the Examples. Further, the early stage seed-specific and endosperm-preferential expression capacity of the identified or generated fragments of the promoters of the invention can be conveniently tested by operably linking such DNA molecules to a nucleotide sequence encoding an easy scorable marker, e.g. a beta-glucuronidase gene, introducing such a chimeric gene into a plant and analyzing the expression pattern of the marker in seeds at different developmental stages as compared with the expression pattern of the marker in other parts of the plant or in seeds at other developmental stages. Other candidates for a marker (or a reporter gene) are chloramphenicol acetyl transferase (CAT) and proteins with fluorescent properties, such as green fluorescent protein (GFP) from Aequora victoria, or proteins with luminescent properties such as the Renilla luciferase or the bacterial lux operon. To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins, which are easily measured, including, but not limited to, chloramphenicol acetyl transferase (CAT), beta-glucuronidase (GUS), green fluorescent protein (GFP), beta-galactosidase (beta-GAL), and luciferase. The expression cassette containing the reporter gene under the control of the promoter can be introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography. The level of enzyme activity corresponds to the amount of enzyme that was made, which in turn reveals the level of expression and the early stage seed-specific and endosperm preferential functionality from the promoter or promoter fragment of interest. This level of expression can also be compared to other promoters to determine the relative strength of the promoter under study. Once activity and functionality is confirmed, additional mutational and/or deletion analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then again introduced in cells and their activity and/or functionality determined.

The activity or strength of a promoter may be measured in terms of the amount of mRNA or protein accumulation it specifically produces, relative to the total amount of mRNA or protein. The promoter preferably expresses an operably linked nucleic acid sequence at a level greater than about 0.001%, about 0.002%, more preferably greater than about 0.005% of the total mRNA. Alternatively, the activity or strength of a promoter may be expressed relative to a well-characterized promoter (for which transcriptional activity was previously assessed).

It will herein further be clear that equivalent early stage seed-specific and endosperm-preferential promoters can be isolated from other plants. To this end, equivalent promoters can be isolated using the coding sequences of the genes driven by the promoters of any one of SEQ ID NOs: 2 to 7 to screen a genomic library (e.g. by hybridization or in silico) of a crop of interest. When sufficient identity between the coding sequences is obtained (for example, higher than 80% homology) then promoter regions can be isolated upstream of the orthologous genes.

Suitable to the invention are nucleic acids comprising early stage seed-specific and endosperm preferential promoter activity which comprise a nucleotide sequence having at least 40%, at least 50%, or at least 60%, or at least 70%, or at least 80%, or at least 85%, or at least 90%, or at least 95%, or at least 98% sequence identity to the herein described promoters and promoter regions or functional fragments thereof and are also referred to as variants. The term “variant” with respect to the transcription regulating nucleotide sequences SEQ ID NOs: 2 to 7 of the invention is intended to mean substantially similar sequences. Naturally occurring allelic variants such as these can be identified with the use of well-known molecular biology techniques, as, for example, with polymerase chain reaction (PCR) and hybridization techniques as herein outlined before. Variant nucleotide sequences also include synthetically derived nucleotide sequences, such as those generated, for example, by using site-directed mutagenesis of any one of SEQ ID NOs: 2 to 7. Generally, nucleotide sequence variants of the invention will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence or a functional fragment thereof. Derivatives of the DNA molecules disclosed herein may include, but are not limited to, deletions of sequence, single or multiple point mutations, alterations at a particular restriction enzyme site, addition of functional elements, or other means of molecular modification which may enhance, or otherwise alter promoter expression. Techniques for obtaining such derivatives are well-known in the art (see, for example, J. F. Sambrook, D. W. Russell, and N. Irwin (2000) Molecular Cloning: A Laboratory Manual, 3^{rd} edition Volumes 1, 2, and 3. Cold Spring Harbor Laboratory Press). For example, one of ordinary skill in the art may delimit the functional elements within the promoters disclosed herein and delete any non-essential elements. Functional elements may be modified or combined to increase the utility or expression of the sequences of the invention for any particular application. Those of skill in the art are familiar with the standard resource materials that describe specific conditions and procedures for the construction, manipulation, and isolation of macromolecules (e.g., DNA molecules, plasmids, etc.), as well as the generation of recombinant organisms and the screening and isolation of DNA molecules. As used herein, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S. Introduction to Computational Biology: Maps, sequences and genomes. Chapman & Hall. London (1995), the homology alignment algorithm of Needleman and Wunsch (J. Mol. Biol., 48:443-453 (1970), the search for similarity method of Pearson and Lipman (Proc. Natl. Acad. Sci., 85:2444 (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence.

A nucleic acid comprising a nucleotide sequence having at least 80% sequence identity to any one of SEQ ID NOs: 2 to 7 can thus be a nucleic acid comprising a nucleotide sequence having at least 80%, or at least 85%, or at least 90%, or at least 95%, or at least 98%, or 100% sequence identity to any one of SEQ ID NOs: 2 to 7.

A “functional fragment” of a nucleic acid comprising early stage seed-specific and endosperm-preferential promoter denotes a nucleic acid comprising a stretch of the nucleic acid sequences of any one of SEQ ID NOs: 2 to 7, or of the nucleic acid having at least 80% sequence identity to any one of SEQ ID NOs: 2 to 7 which still exerts the desired function, i.e. which has early stage seed-specific and endosperm preferential promoter activity. Assays for determining early stage seed-specific and endosperm-preferential promoter activity are provided herein. Preferably, the functional fragment of the early stage seed-specific and endosperm-preferential promoter contains the conserved promoter motifs, such as, for example, conserved promoter motifs as described in DoOP (doop.abc.hu, databases of Orthologous Promoters, Barta E. et al (2005) Nucleic Acids Research Vol. 33, D86-D90). A functional fragment may be a fragment of at least about 900 bp from the transcription start site.

A nucleic acid comprising the nucleotide sequence of any one of SEQ ID NO: 2 to SEQ ID NO: 7 which further comprises insertion, deletion, substitution of at least 1 nucleotide up to 20 nucleotides, at least 1 nucleotide up to 15 nucleotides, at least 1 nucleotide up to 10 nucleotides, at least 1 nucleotide up to 5 nucleotides, at least 1 nucleotide up to 4 nucleotides, at least 1 nucleotide up to 3 nucleotides, or even at least 1 nucleotide up to 2 nucleotides may cover at least about 900 bp from the transcription start site.

A number of consensus elements (motifs) were identified on the promoter sequences disclosed herein.

Variants of the promoter described herein include those which comprise the identified motifs—motif 1 (SEQ ID NO: 20), motif 2 (SEQ ID NO: 21), motif 3 (SEQ ID NO: 22), motif 4 (SEQ ID NO: 23), motif 5 (SEQ ID NO: 24), motif 6 (SEQ ID NO: 25), motif 7 (SEQ ID NO: 26), motif 8 (SEQ ID NO: 27), motif 9 (SEQ ID NO: 28), motif 10 (SEQ ID NO: 29), motif 11 (SEQ ID NO: 30), motif 12 (SEQ ID NO: 31), motif 13 (SEQ ID NO: 32), motif 14 (caagaaa), motif 15 (SEQ ID NO: 33), motif 16 (SEQ ID NO: 34), motif 17 (SEQ ID NO: 35), motif 18 (SEQ ID NO: 36), motif 19 (SEQ ID NO: 37), motif 20 (SEQ ID NO: 38), motif 21 (SEQ ID NO: 39), motif 22 (SEQ ID NO: 40), motif 23 (SEQ ID NO: 41), motif 24 (SEQ ID NO: 42), motif 25 (SEQ ID NO: 43), motif 26 (taagccaa), and/or motif 27 (tttrgmgaa)—but have otherwise been modified to delete nucleotide stretches within the sequence which are not needed for the promoter to be functional in seed-specific and endosperm-preferential manner. For example, any nucleotide stretch located between the motifs and /or between the transcriptional start and the first motif may be at least partially deleted to result in a shorter nucleotide sequence than the about 1 kb sequence of any one of SEQ ID NO: 2 to SEQ ID NO: 7.

“Isolated nucleic acid”, used interchangeably with “isolated DNA” as used herein refers to a nucleic acid not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

A further embodiment provides a recombinant gene comprising the nucleic acid according to the invention operably linked to a heterologous nucleic acid sequence encoding an expression product of interest, and optionally a transcription termination and polyadenylation sequence, preferably a transcription termination and polyadenylation region functional in plant cells. In a further embodiment, said expression product of interest an RNA capable of modulating the expression of a gene or is a protein.

The term “expression product” refers to a product of transcription. Said expression product can be the transcribed RNA. It is understood that the RNA which is produced is a biologically active RNA. Said expression product can also be a peptide, a polypeptide, or a protein, when said biologically active RNA is an mRNA and said protein is produced by translation of said mRNA.

Alternatively, the heterologous nucleic acid, operably linked to the promoters of the invention, may also code for an RNA capable of modulating the expression of a gene. Said RNA capable of modulating the expression of a gene can be an RNA which reduces expression of a gene. Said RNA can reduce the expression of a gene for example through the mechanism of RNA-mediated gene silencing.

Said RNA capable of modulating the expression of a gene can be a silencing RNA down-regulating expression of a target gene. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200 and 1000 bp). hpRNA can also be rather small ranging in size from about 30 to about 42 bp, but not much longer than 94 bp (see WO04/073390, herein incorporated by reference). Silencing RNA may also be artificial micro-RNA molecules as described e.g. in WO2005/052170, WO2005/047505 or US 2005/0144667, or ta-siRNAs as described in WO2006/074400 (all documents incorporated herein by reference). Said RNA capable of modulating the expression of a gene can also be an RNA ribozyme.

Said RNA capable of modulating the expression of a gene can modulate, preferably down-regulate, the expression of other genes (i.e. target genes) comprised within the seeds or even of genes present within a pathogen or pest that feeds upon the seeds of the transgenic plant such as a virus, fungus, insect, bacteria.

The nucleic acid sequence heterologous to the promoters according to the invention may generally be any nucleic acid sequence effecting increased, altered (e.g. in a different organ) or reduced level of transcription of a gene for which such expression modulation is desired. The nucleic acid sequence can for example encode a protein of interest. Exemplary genes for which an increased or reduced level of transcription may be desired in the seeds are e.g. nucleic acids that can provide an agriculturally or industrially important feature in seeds. Suitable heterologous nucleic acid sequences of interest include nucleic acids modulating expression of genes conferring resistance to diseases, stress tolerance genes, genes involved in at different stages of fatty acid biosynthesis or degradation, in acyl editing, in storage compound storage or breakdown, genes encoding epoxidases, hydroxylases, cytochrome P450 mono-oxygenases, desaturases, tocopherol biosynthetic enzymes, carotenoid biosynthesis enzymes, amino acid biosynthetic enzymes, steroid pathway enzymes, starch branching enzymes, genes encoding proteins involved in starch synthesis, glycolysis, carbon metabolism, oxidative pentose phosphate cycle, protein synthesis, organelle organization and biogenesis, DNA metabolism, DNA replication, cell cycle, cell organization and biogenesis, cell proliferation, chromosome organization and biogenesis, microtubule-based processes, microtubule-based movement, cytoskeleton-dependent intracellular transport, cytoskeleton organization and biogenesis, chromatin assembly or disassembly, DNA-dependent DNA replication, chromosome organization and biogenesis, DNA packaging, establishment and/or maintenance of chromatin architecture, regulation of progression through the cell cycle, regulation of the cell cycle, nucleobase, nucleoside, nucleotide and nucleic acid metabolism, chromatin assembly, macromolecule biosynthesis, intracellular transport, establishment of cellular localization, cellular localization, nucleosome assembly, macromolecule metabolism, or M-phase; genes involved in secondary metabolism or genes involved in seed and/or seed coat architecture.

Genes involved in the fatty acid biosynthesis or degradation include but are not limited to genes encoding an acyl-CoA synthetase, a glycerol-phosphate acyltransferase, an 0-acyltransferase, a lyso-phosphatidic acid acyltransferase, a phosphatidic acid phosphatase, a diacylglycerol acyltransferase, an oleate desaturases, a linoleate desaturases, an acyl-CoA hydroxylase, an acyl-lipid hydroxylase, a fatty acid epoxidase, a phospholipid:sterol acyltransferase, a phospholipid:diacylglycerol acyltransferase, a diacylglycerol transacylase, a lysophosphatidylcholine acyltransferase, a phosphatidylcholine:diacylglycerol cholinephosphotransferase, an acyl-CoA elongase, an acyl-lipid elongase, a phosphatidylglycerol-phosphate synthetase, a phosphatidylglycerol-phosphate phosphatase, a CDP-diacylglycerol synthetase, a phosphatidylinositol synthase, a phosphatidylserine synthase, a choline kinase, an ethanolamine kinase, a CDP-choline synthetase, a CDP-ethanolamine synthetase, a phosphatidylserine decarboxylase, a lipoxygenase, a phospholipase, a lipase, a carboxylesterase, a fatty alcohol reductase, a wax ester synthase, a bifunctional acyltranferases/wax synthase, a ketoacyl-CoA synthase, a ketoacyl-CoA reductase, a hydroxylacyl-CoA dehydrase, an enoyl-CoA reductase, an alcohol-forming fatty acyl-CoA reductase, an aldehyde-forming fatty acyl-CoA reductase, an aldehyde decarbonylase, a wax ester hydrolase, a glycerol-3-P-dehydrogenase, a CDP-choline:1,2-diacylglycerol cholinephosphotransferase, an oxidase, a ketosphinganine reductase, a ceramide synthase, an acylglycerophosphorylcholine acyltransferase, an acylglycerol-phosphate acyltransferase, a phosphoethanolamine N-methyltransferase, a ceramide sphingobase desaturase, a glucosylceramide synthase, a acyl-ceramide synthase, a triacylglycerol lipase, a monoacylglycerol lipase, an acyl-CoA oxidase, an hydroxyacyl-CoA dehydrogenase, a dienoyl-CoA reductase, a fatty acid omega-alcohol oxidase, a monoacylglycerol lipase, an acyl-CoA oxidase, a hydroxyacyl-CoA dehydrogenase, a dienoyl-CoA reductase, a fatty acid omega-alcohol oxidase, a fatty acid/acyl-CoA transporter, a acyl-CoA dehydrogenase, a diacylglycerol-phosphate kinase, a lysophosphatidic acic phosphatase, a peroxygenase; a Δ4-desaturase; a Δ5-desaturase, a Δ6-desaturase; a Δ9-desaturase, a Δ12-desaturase or a Δ15-desaturase.

Genes involved in cell proliferation include but are not limited to genes encoding Dal (Li et al., 2008, Genes Dev 22:1331, WO2015/067943), Da2, EOD1 or EOD3 (WO2015/022192, PCT/GB/2013/050072).

A “transcription termination and polyadenylation region” as used herein is a sequence that drives the cleavage of the nascent RNA, whereafter a poly(A) tail is added at the resulting RNA 3′ end, functional in plant cells. Transcription termination and polyadenylation signals functional in plant cells include, but are not limited to, 3′nos, 3′35S, 3′his and 3′g7.

The term “protein” interchangeably used with the term “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

The term “heterologous” refers to the relationship between two or more nucleic acid or protein sequences that are derived from different sources. For example, a promoter is heterologous with respect to an operably linked DNA region, such as a coding sequence if such a combination is not normally found in nature. In addition, a particular sequence may be “heterologous” with respect to a cell or organism into which it is inserted (i.e. does not naturally occur in that particular cell or organism).

The term “recombinant gene” refers to any gene that contains: a) DNA sequences, including regulatory and coding sequences that are not found together in nature, or b) sequences encoding parts of proteins not naturally adjoined, or c) parts of promoters that are not naturally adjoined. Accordingly, a recombinant gene may comprise regulatory sequences and coding sequences that are derived from different sources, or comprise regulatory sequences, and coding sequences derived from the same source, but arranged in a manner different from that found in nature.

Any of the promoters and heterologous nucleic acid sequences described above may be provided in a recombinant vector. A recombinant vector typically comprises, in a 5′ to 3′ orientation: a promoter to direct the transcription of a nucleic acid sequence and a nucleic acid sequence. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired. The wording “5′ UTR” refers to the untranslated region of DNA upstream, or 5′ of the coding region of a gene and “3′ UTR” refers to the untranslated region of DNA downstream, or 3′ of the coding region of a gene. Means for preparing recombinant vectors are well known in the art. Methods for making recombinant vectors particularly suited to plant transformation are described in U.S. Pat. No. 4,971,908, U.S. Pat. No. 4,940,835, U.S. Pat. No. 4,769,061 and U.S. Pat. No. 4,757,011. Typical vectors useful for expression of nucleic acids in higher plants are well known in the art and include vectors derived from the tumor-inducing (Ti) plasmid of Agrobacterium tumefaciens. One or more additional promoters may also be provided in the recombinant vector. These promoters may be operably linked, for example, without limitation, to any of the nucleic acid sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Also, promoters which function in bacteria, yeast, and plants are all well taught in the art. The additional promoters may also be selected on the basis of their regulatory features. Examples of such features include enhancement of transcriptional activity, inducibility, tissue specificity, and developmental stage-specificity.

The recombinant vector may also contain one or more additional nucleic acid sequences. These additional nucleic acid sequences may generally be any sequences suitable for use in a recombinant vector. Such nucleic acid sequences include, without limitation, any of the nucleic acid sequences, and modified forms thereof, described above. The additional structural nucleic acid sequences may also be operably linked to any of the above described promoters. The one or more structural nucleic acid sequences may each be operably linked to separate promoters. Alternatively, the structural nucleic acid sequences may be operably linked to a single promoter (i.e., a single operon).

Yet another embodiment provides a host cell, such as an E. coli cell, an Agrobacterium cell, a yeast cell, or a plant cell, comprising the isolated nucleic acid according to the invention, or the recombinant gene according to the invention.

Other nucleic acid sequences may also be introduced into the host cell along with the promoter and structural nucleic acid sequence, e. g. also in connection with the vector of the invention. These other sequences may include 3′ transcriptional terminators, 3′ polyadenylation signals, other untranslated nucleic acid sequences, transit or targeting sequences, selectable markers, enhancers, and operators. Preferred nucleic acid sequences of the present invention, including recombinant vectors, structural nucleic acid sequences, promoters, and other regulatory elements, are described above.

In further embodiments, a plant and a plant cell are provided comprising the recombinant gene according to the invention. In yet a further embodiment, a plant is provided comprising at least two recombinant genes according to the invention, wherein the nucleic acid comprising early stage seed-specific and endosperm preferential promoter activity according to the invention and, optionally, the heterologous nucleic acid sequence operably linked thereto, are different in each recombinant gene. Yet a further embodiment provides seeds obtainable from the plant according to the invention. In another embodiment, the plants or seeds according to the invention are seed crop plants or seeds.

The plant cell or plant comprising the recombinant gene according to the invention can be a plant cell or a plant comprising a recombinant gene of which either the promoter, or the heterologous nucleic acid sequence operably linked to said promoter, are heterologous with respect to the plant cell. Such plant cells or plants may be transgenic plant in which the recombinant gene is introduced via transformation. Alternatively, the plant cell of plant may comprise the promoter according to the invention derived from the same species operably linked to a nucleic acid which is also derived from the same species, i.e. neither the promoter nor the operably linked nucleic acid is heterologous with respect to the plant cell, but the promoter is operably linked to a nucleic acid to which it is not linked in nature. A recombinant gene can be introduced in the plant or plant cell via transformation, such that both the promoter and the operably linked nucleotide are at a position in the genome in which they do not occur naturally. Alternatively, the promoter according to the invention can be integrated in a targeted manner in the genome of the plant or plant cell upstream of an endogenous nucleic acid encoding an expression product of interest, i.e. to modulate the expression pattern of an endogenous gene. The promoter that is integrated in a targeted manner upstream of an endogenous nucleic acid can be integrated in cells of a plant species from which it is originally derived, or in cells of a heterologous plant species. Alternatively, a heterologous nucleic acid can be integrated in a targeted manner in the genome of the plant or plant cell downstream of the promoter according to the invention, such that said heterologous nucleic acid is expressed seed-specifically and endosperm-preferentially. Said heterologous nucleic acid is a nucleic acid which is heterologous with respect to the promoter, i.e. the combination of the promoter with said heterologous nucleic acid is not normally found in nature. Said heterologous nucleic acid may be a nucleic acid which is heterologous to said plant species in which it is inserted, but it may also naturally occur in said plant species at a different location in the plant genome. Said promoter or said heterologous nucleic acid can be integrated in a targeted manner in the plant genome via targeted sequence insertion, using, for example, the methods as described in WO2005/049842.

Plants comprising at least two recombinant genes according to the invention wherein the nucleic acid comprising seed-specific and endosperm-preferential promoter activity is different in each recombinant gene are, for example, plants comprising a first recombinant gene comprising a nucleotide sequence having at least 80% sequence identity to SEQ ID NO: 2 or a functional fragment thereof, and a second recombinant gene comprising a nucleotide sequence having at least 80% sequence identity to any one of SEQ ID NO: 3 to SEQ ID NO: 7 or a functional fragment thereof. It will be clear that, when the first recombinant gene comprises a nucleotide sequence having at least 80% sequence identity to SEQ ID NO: x or a functional fragment thereof, wherein SEQ ID NO: x is selected from any one of SEQ ID NO: 2 to SEQ ID NO: 7, the second recombinant gene may comprise a nucleotide sequence having at least 80% sequence identity to any one of the sequences according to the invention or a functional fragment thereof, except to SEQ ID NO: x. Said plants are suitable to express different genes with the same tissue-specificity, however without the negative features associated with the repeated use of one promoter, such as gene silencing or recombination of a vector comprising the recombinant genes. The at least two recombinant genes according to the invention may be present at one locus in the genome of said plant, and may be derived from the same transforming DNA molecule.

Plants according to the invention may comprise one or more recombinant genes according to the invention, but may in addition contain a recombinant gene comprising a nucleic acid comprising promoter activity which is preferential or specific to other plant tissues, such as apical meristem, flower buds, cotyledons, flowers, pods, roots, and leaves or other seed developmental stages, operably linked to a nucleic acid sequence encoding an expression product of interest. The recombinant gene according to the invention and the recombinant gene comprising a nucleic acid comprising another promoter activity may be present at one locus and may be derived from the same transforming DNA molecule.

Yet another embodiment provides a method of producing a transgenic plant comprising the steps of (a) introducing or providing the recombinant gene according to the invention to a plant cell to create transgenic cells; and (b) regenerating transgenic plants from said transgenic cell.

“Introducing” in connection with the present application relates to the placing of genetic information in a plant cell or plant by artificial means. This can be effected by any method known in the art for introducing RNA or DNA into plant cells, protoplasts, calli, roots, tubers, seeds, stems, leaves, seedlings, embryos, pollen and microspores, other plant tissues, or whole plants. “Introducing” also comprises stably integrating into the plant's genome. Introducing the recombinant gene can be performed by transformation.

The term “transformation” herein refers to the introduction (or transfer) of nucleic acid into a recipient host such as a plant or any plant parts or tissues including plant cells, protoplasts, calli, roots, tubers, seeds, stems, leaves, seedlings, embryos and pollen. Plants containing the transformed nucleic acid sequence are referred to as “transgenic plants”. Transformed, transgenic and recombinant refer to a host organism such as a plant into which a heterologous nucleic acid molecule (e.g. an expression cassette or a recombinant vector) has been introduced. The nucleic acid can be stably integrated into the genome of the plant.

As used herein, the phrase “transgenic plant” refers to a plant having an introduced nucleic acid stably introduced into a genome of the plant, for example, the nuclear or plastid genomes. In other words, plants containing transformed nucleic acid sequence are referred to as “transgenic plants”. Transgenic and recombinant refer to a host organism such as a plant into which a heterologous nucleic acid molecule (e.g. the promoter, the chimeric gene or the vector as described herein) has been introduced. The nucleic acid can be stably integrated into the genome of the plant.

Transformation methods are well known in the art and include Agrobacterium-mediated transformation. Agrobacterium-mediated transformation of cotton has been described e.g. in U.S. Pat. No. 5,004,863, in U.S. Pat. No. 6,483,013 and WO2000/71733. Plants may also be transformed by particle bombardment: Particles of gold or tungsten are coated with DNA and then shot into young plant cells or plant embryos. This method also allows transformation of plant plastids. Viral transformation (transduction) may be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004. Further suitable methods well-known in the art are microinjection, electroporation of intact cells, polyethyleneglycol-mediated protoplast transformation, electroporation of protoplasts, liposome-mediated transformation, silicon-whiskers mediated transformation etc. Said transgene may be stably integrated into the genome of said plant cell, resulting in a transformed plant cell. The transformed plant cells obtained in this way may then be regenerated into mature fertile transformed plants.

Further provided is a method of effecting early stage seed-specific and endosperm preferential expression of a nucleic acid comprising introducing the recombinant gene according to the invention into the genome of a plant, or providing the plant according to the invention. Also provided is a method for altering seed properties of a plant or to produce a commercially relevant product in a plant, comprising introducing the recombinant gene according to the invention into the genome of a plant, or providing the plant according to the invention. In another embodiment, said plant is a seed crop plant.

“Seed properties” as used herein are properties of the seed. Seed properties can, for example, be seed yield, seed storage compound production, seed compound accumulation, seed nutrient accumulation; seed micronutrient accumulation; seed storage compound quality, seed compound composition, seed quality, biotic stress tolerance such as disease tolerance, abiotic stress tolerance, herbicide tolerance, seed dormancy, seed imbibition, seed germination, seed vigor. Seed storage compounds can, for example, be, seed oil, seed starch, or seed protein.

Seed properties may be modulated by modulating metabolic pathways, such as starch metabolism, sugar metabolism, inositol phosphate metabolism, glycolysis, amino acid biosynthesis, carbon metabolism, nucleotide metabolism, oxidative pentose phosphate cycle, fatty acid biosynthesis, protein synthesis, or phytate metabolism, and modulating secondary metabolism pathways. Another example is the methyl recycling metabolic activity impacting chromatin remodeling, phospholipid biosynthesis and cell wall lignification. Such metabolic pathways can be modulated by, for example, overexpressing or down-regulating a gene involved in one or more of the metabolic pathways using the early stage seed-specific and endosperm preferential promoter according to the invention.

Yield as used herein can comprise yield of the plant or plant part which is harvested, such as seed, including seed oil content, seed protein content, seed weight, seed number. Increased yield can be increased yield per plant, and increased yield per surface unit of cultivated land, such as yield per hectare. Yield can be increased by modulating, for example, by increasing seed size or oil content or indirectly by increasing the tolerance to biotic and abiotic stress conditions and decreasing seed abortion.

Quality as used herein can comprise quality of the seed or grain such as beneficial carbohydrate composition or level, beneficial amino acid composition or level, beneficial fatty acid composition or level, nutritional value, seed and fiber content.

Abiotic stress tolerance as used herein can comprise resistance to environmental stress factors such as drought, extreme (high or low) temperatures.

Biotic stress tolerance as used herein can comprise pest resistance, such as resistance or fungal, bacterial, bacterial or viral pathogens or insects.

Also provided is the use of the isolated nucleic acid according to the invention to regulate expression of an operably linked nucleic acid in a plant, and the use of the isolated nucleic acid according to the invention, or the recombinant gene according to the invention to alter seed properties of a plant or to produce a commercially relevant product in a plant. In a further embodiment, said plant is a trait as used herein refers to beneficial properties of the plant, such as commercially beneficial properties of a plant.

Also provided is the use of the isolated nucleic acid according to the invention to identify other nucleic acids comprising early stage seed-specific and endosperm preferential promoter activity.

Other nucleic acids comprising early stage seed-specific and endosperm preferential promoter activity can be identified using methods known in the art. Such nucleotide sequence may be identified and isolated by hybridization under stringent conditions using as probes a nucleic acid comprising any one of the nucleotide sequences of any one of SEQ ID NOs: 2 to 7 or part thereof. Other nucleic acids comprising early stage seed-specific and endosperm preferential promoter activity may also be obtained by DNA amplification using oligonucleotides specific for the sequences according to the invention as primers, such as but not limited to oligonucleotides comprising or consisting of about 20 to about 50 consecutive nucleotides from any one of the nucleotide sequences of any one of SEQ ID NOs: 2 to 7 or its complement. Other nucleic acids comprising early stage seed-specific and endosperm preferential promoter activity can be identified in silico using Basic Local Alignment Search Tool (BLAST) homology search with other nucleotide or amino acid sequences. Functionality of the identified nucleic acids comprising early stage seed-specific and endosperm preferential promoter activity can be validated using the methods described herein. Other nucleic acids comprising early stage seed-specific and endosperm preferential promoter activity may also be identified by identification of gene sequences orthologous to the gene sequences of the endogenous coding sequences of the genes driven by the promoters of the invention, and isolating the promoter sequences upstream of these orthologous homologous coding sequences.

The promoters according to the invention can further be used to create hybrid promoters, i.e. promoters containing (parts of) one or more of the promoters(s) of the current invention and (parts of) other promoter which can be newly identified or known in the art. Such hybrid promoters may have optimized tissue specificity or expression level.

Yet another embodiment provides a method of producing food, feed, or an industrial product comprising (a) obtaining the plant or a part thereof, according to the invention; and (b) preparing the food, feed or industrial product from the plant or part thereof. In another embodiment, said food or feed is oil, meal, grain, starch, flour or protein, or said industrial product is biofuel, fiber, industrial chemicals, a pharmaceutical or a nutraceutical.

A “seed crop” or “seed crop plant” as used herein is a crop grown for its seeds or material derived from the seeds. Examples of seed crops are rice, maize, wheat, barley, millet, rye, oats, camelina, crambe, Linum, castor bean, calendula, safflower, sunflower, soybean, cotton, or Brassica species, such as Brassica napus, Brassica juncea, Brassica carinata, Brassica rapa, Brassica oleracea, and Brassica nigra.

“Brassicaceae” or “Brassicaceae plant” as used herein refers to plants belonging to the family of Brassicaceae plants, also called Cruciferae or mustard family. Examples of Brassicaceae are, but are not limited to, Brassica species, such as Brassica napus, Brassica oleracea, Brassica rapa, Brassica carinata, Brassica nigra, and Brassica juncea; Raphanus species, such as Raphanus caudatus, Raphanus raphanistrum, and Raphanus sativus; Matthiola species; Cheiranthus species; Camelina species, such as Camelina sativa; Crambe species, such as Crambe abyssinica and Crambe hispanica; Eruca species, such as Eruca vesicaria; Sinapis species such as Sinapis alba; Diplotaxis species; Lepidium species; Nasturtium species; Orychophragmus species; Armoracia species, Eutrema species; Lepidium species; and Arabidopsis species.

Said Brassicaceae plant can be a Brassica plant. “Brassica plant” refers to allotetraploid or amphidiploid Brassica napus (AACC, 2n=38), Brassica juncea (AABB, 2n=36), Brassica carinata (BBCC, 2n=34), or to diploid Brassica rapa (syn. B. campestris) (AA, 2n=20), Brassica oleracea (CC, 2n=18) or Brassica nigra (BB, 2n=16).

Crop plants of the Brassica species are, for example, Brassica napus, Brassica juncea, Brassica carinata, Brassica rapa (syn. B. campestris), Brassica oleracea or Brassica nigra.

The plants according to the invention may additionally contain an endogenous or a transgene, which confers herbicide resistance, such as the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola. Further, the plants according to the invention may additionally contain an endogenous or a transgene which confers increased oil content or improved oil composition, such as a 12:0 ACP thioesteraseincrease to obtain high laureate, which confers pollination control, such as such as barnase under control of an anther-specific promoter to obtain male sterility, or barstar under control of an anther-specific promoter to confer restoration of male sterility, or such as the Ogura cytoplasmic male sterility and nuclear restorer of fertility.

The plants or seeds of the plants according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34.

Insecticides: Acetamiprid, Aldicarb, Azadirachtin, Carbofuran, Chlorantraniliprole (Rynaxypyr), Clothianidin,

Cyantraniliprole (Cyazypyr), (beta-)Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram,Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methylyl H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1 -yl]methyl}-1H-pyrazole-5-carboxamide, 1-{2-fluoro-4-methyl-5-[(2,2,2-trifluorethyl)sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl)ethanimidamide, Bacillus firmus, Bacillus firmus strain 1-1582, Bacillus subtilis, Bacillus subtilis strain GB03, Bacillus subtilis strain QST 713, Metarhizium anisopliae F52.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention as well as plant cells generated according to the methods of the invention, may be non-propagating cells.

The obtained plants according to the invention can be used in a conventional breeding scheme to produce more plants with the same characteristics or to introduce the same characteristic in other varieties of the same or related plant species, or in hybrid plants. The obtained plants can further be used for creating propagating material. Plants according to the invention can further be used to produce gametes, seeds (including crushed seeds and seed cakes), seed oil, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

Furthermore, the disclosed invention is expected to yield similar results in other seed crop plant species. Particularly, it is expected to drive early stage seed-specific and endosperm preferential expression in soybean. It is also expected to drive early stage seed-specific and endosperm preferential expression in wheat. The disclosed promoter may lead to a early stage seed-specific and endosperm preferential expression in cotton.

The sequence listing contained in the file named “BCS16-2002_ST25.txt”, which is 35 kilobytes (size as measured in Microsoft Windows®), contains 43 sequences SEQ ID NO: 1 through SEQ ID NO: 43 is filed herewith by electronic submission and is incorporated by reference herein.

In the description and examples, reference is made to the following sequences:

### SEQUENCES

SEQ ID NO: 1: nucleotide sequence of the T-DNA Penodl 110 BnC::GUS.

SEQ ID NO: 2: nucleotide sequence of the promoter Penodl 110 BnC.

SEQ ID NO: 3: nucleotide sequence of the promoter Penodl 110 BnA.

SEQ ID NO: 4: nucleotide sequence of the promoter Penodl 110 Bo.

SEQ ID NO: 5: nucleotide sequence of the promoter Penodl 110 Br.

SEQ ID NO: 6: nucleotide sequence of the promoter Penodl 110 BjB.

SEQ ID NO: 7: nucleotide sequence of the promoter Penodl 110 BjA.

SEQ ID NO: 8: amino acid sequence of ENODL10 BnC.

SEQ ID NO: 9: amino acid sequence of ENODL10 BnA.

SEQ ID NO: 10: amino acid sequence of ENODL10 Bo.

SEQ ID NO: 11: amino acid sequence of ENODL10 Br.

SEQ ID NO: 12: amino acid sequence of ENODL10 BjB.

SEQ ID NO: 13: amino acid sequence of ENODL10 BjA.

SEQ ID NO: 14: nucleotide sequence of the coding sequence of ENODL10 BnC.

SEQ ID NO: 15: nucleotide sequence of the coding sequence of ENODL10 BnA.

SEQ ID NO: 16: nucleotide sequence of the coding sequence of ENODL10 Bo.

SEQ ID NO: 17: nucleotide sequence of the coding sequence of ENODL10 Br.

SEQ ID NO: 18: nucleotide sequence of the coding sequence of ENODL10 BjB.

SEQ ID NO: 19: nucleotide sequence of the coding sequence of ENODL10 BjA.

SEQ ID NO: 20: motif 1.

SEQ ID NO: 21: motif 2.

SEQ ID NO: 22: motif 3.

SEQ ID NO: 23: motif 4.

SEQ ID NO: 24: motif 5.

SEQ ID NO: 25: motif 6.

SEQ ID NO: 26: motif 7.

SEQ ID NO: 27: motif 8.

SEQ ID NO: 28: motif 9.

SEQ ID NO: 29: motif 10.

SEQ ID NO: 30: motif 11.

SEQ ID NO: 31: motif 12.

SEQ ID NO: 32: motif 13.

SEQ ID NO: 33: motif 15.

SEQ ID NO: 34: motif 16.

SEQ ID NO: 35: motif 17.

SEQ ID NO: 36: motif 18.

SEQ ID NO: 37: motif 19.

SEQ ID NO: 38: motif 20.

SEQ ID NO: 39: motif 21.

SEQ ID NO: 40: motif 22.

SEQ ID NO: 41: motif 23.

SEQ ID NO: 42: motif 24.

SEQ ID NO: 43: motif 25.

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1

### Generation of Expression Constructs with the Penodl 10 BnC Promoter of Brassica napus Operably Linked to the GUS Reporter Gene (Penodl 10 BnC::GUS)

The promoter sequence of the Brassica napus enodl10 promoter (SEQ ID NO: 2 or 5′ to 3′ position 139 to 1081 of SEQ ID NO:1) isolated from an in house developed Brassica napus line, the GUS gene (β-glucuronidase) with intron (5′ to 3′ position 1082 to 3082 of SEQ ID NO: 1) and a fragment of the 3′ untranslated region (UTR) of the TL-DNA gene 7 of Agrobacterium tumefaciens octopine (5′ to 3′ position 3139 to 3342 of SEQ ID NO: 1) were assembled in a vector which contains the bar selectable marker cassette (position 3423 to 5933 of SEQ ID NO: 1) to result in the T-DNA Penodl 10 BnC::GUS (SEQ ID NO: 1).

### Example 2

### Generation of Transgenic Plants Comprising the Penodl 10 BnC::GUS

In a next step the recombinant vectors comprising the expression cassette of example 1, i. e. Penodl 10 BnC, were used to stably transform Brassica napus using the hypocotyl transformation protocol.

### Example 3

### In Planta Expression Pattern of Penodl 10 BnC::GUS in Brassica napus

The in planta expression pattern of Penodl 110 BnC::GUS in the different seed tissues and non-seed tissues of Brassica napus seeds was monitored according to the method of Jasik et al. 2011.

No GUS activity was detected in the assessed non-seed tissues, namely young leaves, young stems, flower buds, flowers and pods, thereby confirming the seed-specificity of the selected promoter.

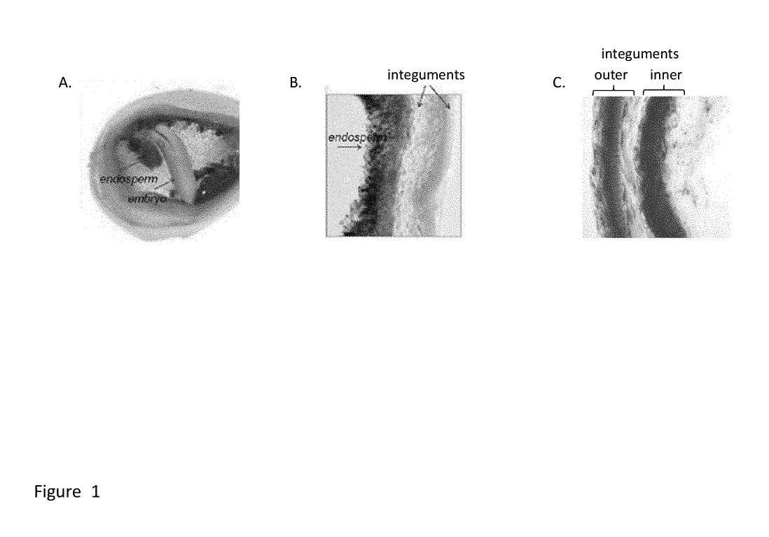

FIG. 1 shows the GUS labelling of the reporter gene in the embryo, endosperm and seed coat at early and late seed developmental stages. The strong staining of the endosperm, at least twice stronger than in the other seed tissues, clearly confirms the endosperm-preferential expression driven by the promoter Penodl 10 BnC (panel A). Close up views on the seed coat at early stage demonstrate that though less pronounced, the transgene expression is also detected in the seed coat, and in both the outer and the inner integument (panel B).Close up views on the seed coat at late stage show that the GUS labelling occurs in the inner integument but not in the outer integument (panel C).

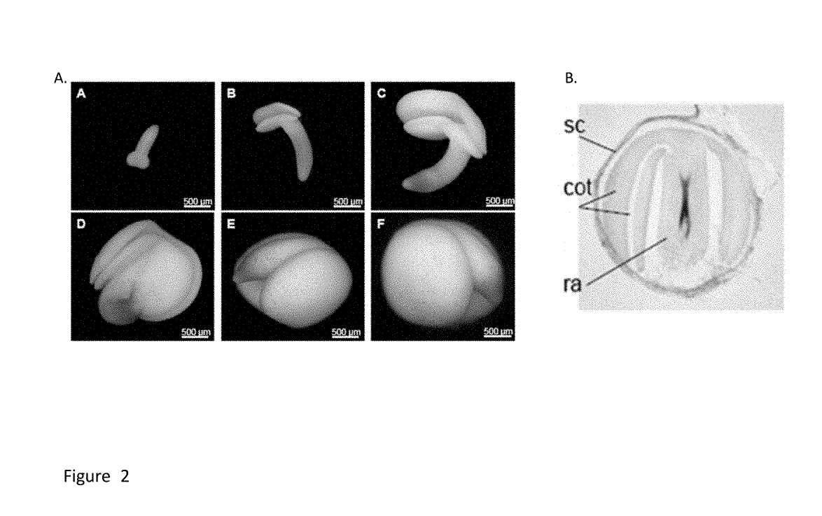

FIG. 2 shows pictures of the stained embryos at different developmental stages. The construct tested lead to weak GUS activity detected in the embryo at early stages of embryo development (panel A, sub-panels A to C). Staining intensity then decreases and preferentially occupies the radicle towards maturation. Although no tissue specificity is visible at the earliest stage, expression becomes progressively restricted to the vasculature in the radicle and hypocotyl in mature embryos (panel B).

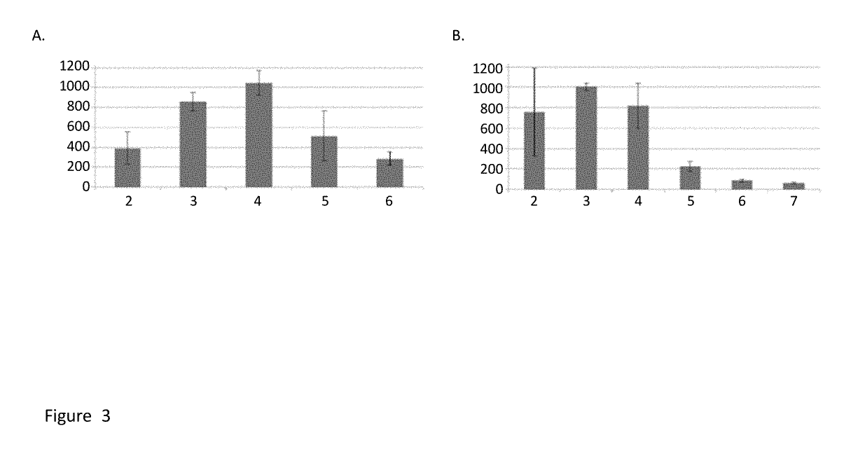

FIG. 3 provides the semi quantitative assessment of the GUS labelling in the seed coat and the embryo of transgenic lines carrying the Penodl 10 BnC::GUS T-DNA. The GUS staining in the seed coat is the strongest during mid-developmental stages (panel A) while in the embryo the labelling is the strongest at early stages and then decreases as the embryo progresses towards maturation (panel B).

### Example 4

### Identification of the Different Brassica napus Copies of ENODL10 and of the Orthologues of ENODL10 in Brassica rapa, Brassica oleracea and Brassica juncea

The sequences of the different Brassica napus copies of ENODL10 as well as their orthologues in Brassica rapa, Brassica oleracea and Brassica juncea were obtained by blasting the coding sequence of the ENODL10 BnC against an in-house database of Brassica napus, Brassica rapa, Brassica oleracea and Brassica juncea sequences.

The nucleotide sequences obtained in this way are given in SEQ ID NO: 14 to SEQ ID NO: 19. These nucleotide sequences were translated into amino acid sequences, given in SEQ ID NO: 8 to SEQ ID NO: 13.

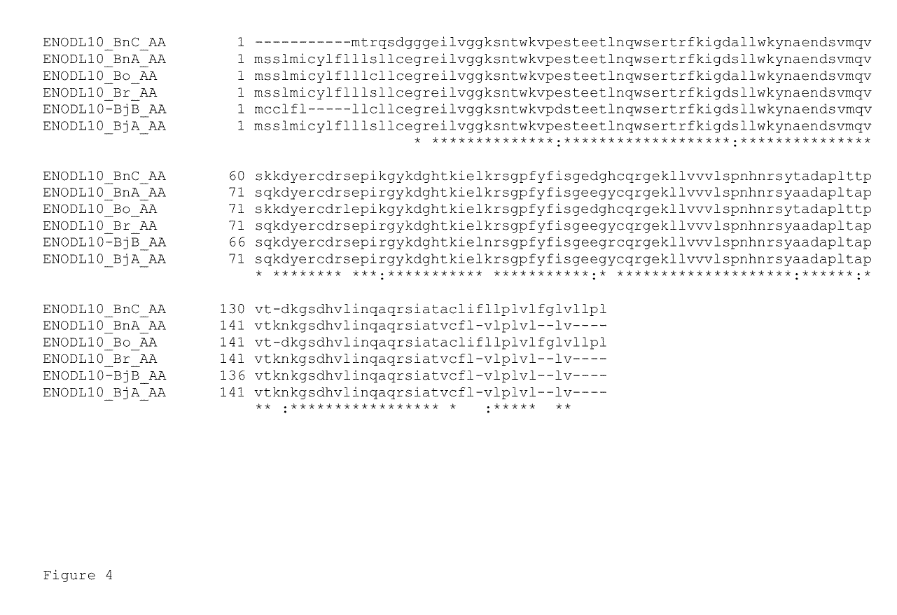

FIG. 4 shows the alignment of the retrieved amino acid sequences. Any two of these sequences share at least 80% sequence homology.

### Example 5

### RNA Isolation from Different Brassica Tissues

The following tissues were isolated from Brassica napus:
a. Apical meristem 33 days after sowing (DAS) (including smallest leaves) (AM33)
b. Big flower buds (>5 mm) 42 DAS (BFB42)
c. Cotyledons (with hypocotyl) 10 DAS (CTYL10)
d. Open flowers 52 DAS (OF52)
e. Pods 14-20 DAS (Pod2)
f. Pods 21-25 DAS (Pod3)
g. Roots 14 DAS (Ro2w)
h. Small flower buds ≤5 mm 42 DAS (SFB42)
i. Seeds 14-20 days after flowering (DAF) (Seed2)
j. Seeds 21-25 DAF (Seed3)
k. Seeds 26-30 DAF (Seed4)
l. Seeds 31-35 DAF (Seed5)
m. Seeds 42 DAF (Seed6)
n. Seeds 49 DAF (Seed7)
o. Stem 14 DAS (St2w)
p. Stem 33 DAS (St5w)
q. Young leaf 33 DAS 3 cm leaf next to apical meristem) (YL33)

The following tissues were isolated from Brassica juncea:
a. Apical meristem 22 days after sowing (DAS) (including smallest leaves) (AM22)
b. Big flower buds (>5 mm) 35 DAS (BFB35)
c. Cotyledons (with hypocotyl) 8 DAS (CTYL8)
d. Open flowers 35 DAS (OF35)
e. Pods 14-20 DAS (Pod2)
f. Pods 21-25 DAS (Pod3)
g. Pods 26-30 DAS (Pod4)
h. Pods 31-35 DAS (Pod5)
i. Roots 14 DAS (Ro2w)
j. Small flower buds ≤5 mm 35 DAS (SFB35)
k. Seeds 14-20 days after flowering (DAF) (Seed2)
l. Seeds 21-25 DAF (Seed3)
m. Seeds 26-30 DAF (Seed4)
n. Seeds 31-35 DAF (Seed5)
o. Seeds 42 DAF (Seed6)
p. Seeds 49 DAF (Seed7)
q. Stem 14 DAS (St2w)
r. Stem 22 DAS (St3w)
s. Young leaf 22 DAS 3 cm leaf next to apical meristem) (YL22)
t. Old leaf 22 DAS (OL22)

The following seed sub-tissues were isolated from Brassica napus:
a. Endosperm, 18 days after flowering (DAF)
b. Endosperm, 24 DAF
c. Embryonic hypocotyl, 18 DAF
d. Embryonic hypocotyl, 24 DAF
e. Embryonic hypocotyl, 28 DAF
f. Embryonic hypocotyl, 32 DAF
g. Embryonic hypocotyl, 46 DAF
h. Embryonic inner cotyledon, 18 DAF
i. Embryonic inner cotyledon, 24 DAF
j. Embryonic inner cotyledon, 28 DAF
k. Embryonic inner cotyledon, 32 DAF
l. Embryonic inner cotyledon, 46 DAF
m. Embryonic outer cotyledon, 18 DAF
n. Embryonic outer cotyledon(inner part), 24 DAF
o. Embryonic outer cotyledon(inner part), 28 DAF
p. Embryonic outer cotyledon(inner part), 32 DAF
q. Embryonic outer cotyledon(inner part), 46 DAF
r. Embryonic outer cotyledon(outer part), 24 DAF
s. Embryonic outer cotyledon(outer part), 28 DAF
t. Embryonic outer cotyledon(outer part), 32 DAF
u. Embryonic outer cotyledon(outer part), 46 DAF

For the isolation of the seed sub-tissues, freshly harvested seeds were frozen at −80° C. and cut into 20 μm sections. Sections were placed on PET-membranes, lyophilized at −20° C., and then used for laser-assisted microdissection (PALM Laser-Microbeam instrument; Bernried/Germany) (for details see Schiebold et al., 2011, Plant Methods 7:19). Up to 5 distinct embryonic tissues plus endosperm were targeted. Tissue dissection was applied to seeds at 18, 24, 28, 32 and 46 DAF, covering the developmental period from onset of storage activity until late maturation. RNA was extracted (purification of total RNA by RNeasy Micro kit; Qiagen) and amplified (C&E version ExpressArt mRNA amplification Nano kit; Amp-tec) as detailed in Schiebold et al. 2011 (supra).

Total RNA from the non-seed sub-tissues was isolated according to standard methods.

In our growth conditions, the correspondence between embryo developmental stages and the selected time points is as follows:
a. Between 10 and 13 DAF: torpedo stage
b. Seed2 or between 14 and 20 DAF: “walking stick” cotyledon stage
c. Seed3 or between 21 and 25 DAF: curled cotyledon stage
d. Seed4 and SeedS or between 26 and 35 DAF: green cotyledon stage
e. Seed6 and Seed7 or after 36 DAF: mature embryo.

### Example 6

### In Silico Eexpression Analyses of the Different Copies of ENODL10 of Brassica napus and Their Orthologues

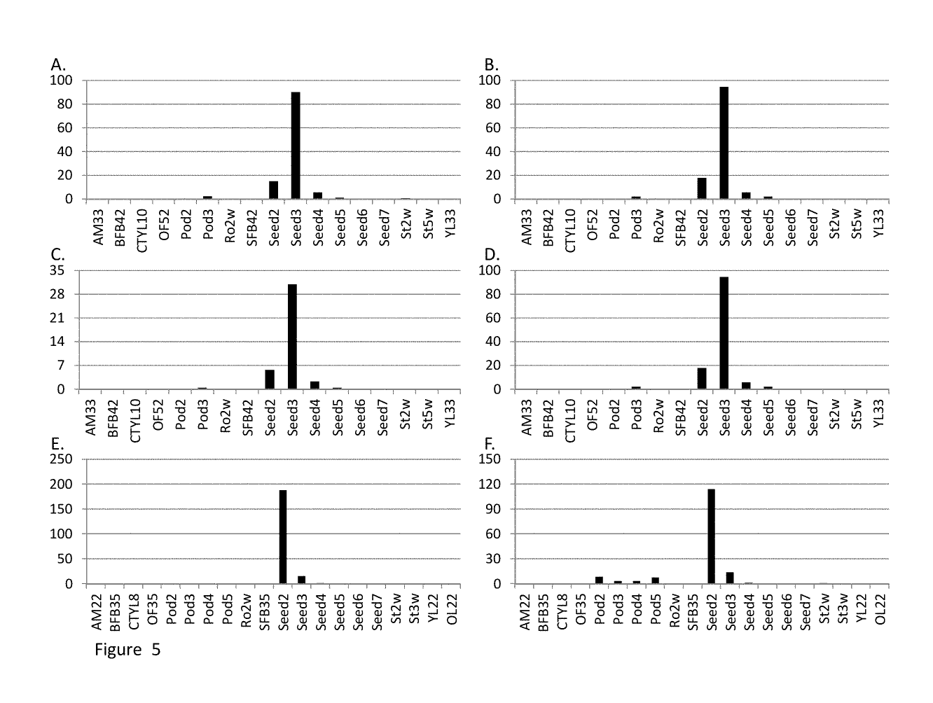

FIG. 5 shows the relative expression levels of the endogenous transcripts of the different Brassica napus (A and B), Brassica oleracea (C), Brassica rapa (D) and Brassica juncea (E and F) copies of ENODL10 in different tissues, as isolated in Example 5.

The ENODL10 BnC transcript (panel A) is abundantly detected in the Seed3 tissues, mildly detected in the Seed2 tissues and only barely detectable in the Seed4, Seed5 and Pod3 tissues. This result confirms, as determined in planta, that Penodl 10 BnC has early stage seed-specific promoter activity.

The ENODL10 BnA transcript (panel B) is abundantly detected in the Seed3 tissues, mildly detected in the Seed2 tissues and only barely detectable in the Seed4, Seed5 and Pod3 tissues. This result indicates that Penodl 10 BnA has early stage seed-specific promoter activity.

The ENODL10 Bo transcript (panel C) is abundantly detected in the Seed3 tissues, mildly detected in the Seed2 tissues and only barely detectable in the Seed4, Seed5 and Pod3 tissues. This result indicates that Penodl 10 Bo has early stage seed-specific promoter activity.

The ENODL10 Br transcript (panel D) is abundantly detected in the Seed3 tissues, mildly detected in the Seed2 tissues and only barely detectable in the Seed4, Seed5 and Pod3 tissues. This result indicates that Penodl 10 Br has early stage seed-specific promoter activity.

The ENODL10 BjB transcript (panel E) is abundantly detected in the Seed2 tissues and only barely detectable in the Seed3, Seed4 and BFB35 tissues. This result indicates that Penodl 10 BjB has early stage seed-specific promoter activity.

The ENODL10 BjA transcript (panel F) is abundantly detected in the Seed2 tissues and only barely detectable in the other tissues. This result indicates that Penodl 10 BjA has early stage seed-specific promoter activity.

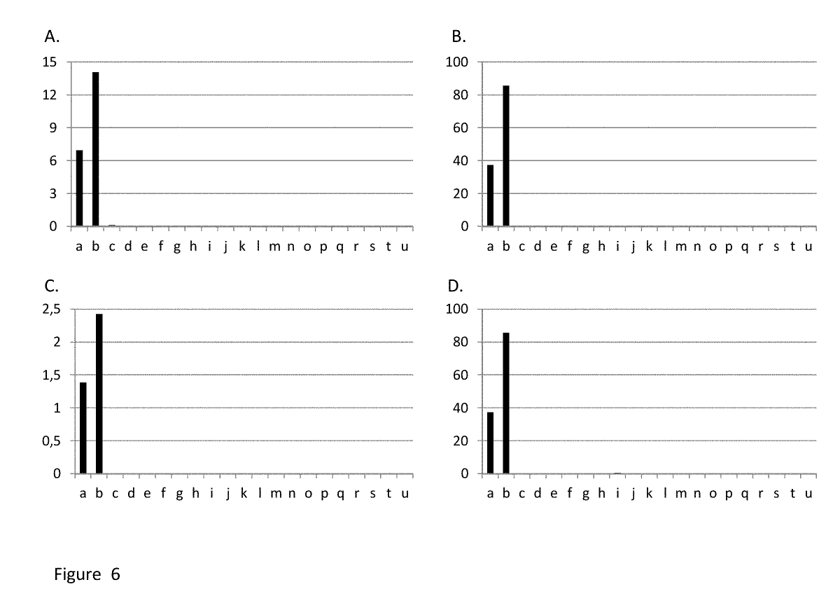

FIG. 6 shows the relative expression levels of the endogenous transcripts of the different Brassica napus (A and B), Brassica oleracea (C) and Brassica rapa (D) copies of ENODL10 in different seed sub-tissues, as isolated in Example 5.

The ENODL10 BnC transcript (panel A) is clearly detected in the endosperm tissues at 18 and 24 DAF. In the embryo, the ENODL10 BnC transcript is only barely detected in the hypocotyl at 18 DAF. This result confirms, as determined in planta, that Penodl 10 BnC has endosperm-preferential promoter activity.

The ENODL10 BnA transcript (panel B) is abundantly detected in the endosperm tissues at 18 and 24 DAF. In the embryo, the ENODL10 BnA transcript is barely detected in the hypocotyl, the inner cotyledon and the outer cotyledon at 18 and 24 DAF. This result indicates that Penodl 10 BnA has endosperm-preferential promoter activity.

The ENODL10 Bo transcript (panel C) is only detected in the endosperm tissues at 18 and 24 DAF. This result indicates that Penodl 10 Bo has endosperm-preferential promoter activity.

The ENODL10 Br transcript (panel D) is abundantly detected in the endosperm tissues at 18 and 24 DAF. In the embryo, the ENODL10 Br transcript is barely detected in the hypocotyl tissues, the inner cotyledon and the outer cotyledon at 18 and 24 DAF. This result indicates that Penodl 10 Br has endosperm-preferential promoter activity.

### Example 7

### Sequence Analysis of the Promoters of the ENODL10 Genes from Brassica rapa, Brassica juncea, Brassica oleracea and Brassica napus

For each ENODL10 gene identified the genomic DNA sequence upstream of the translation start was retrieved from an in-house database of Brassica napus, Brassica rapa, Brassica oleracea and Brassica juncea sequences. The nucleotide sequences obtained in this way are given in SEQ ID NO: 3 to SEQ ID NO: 7.

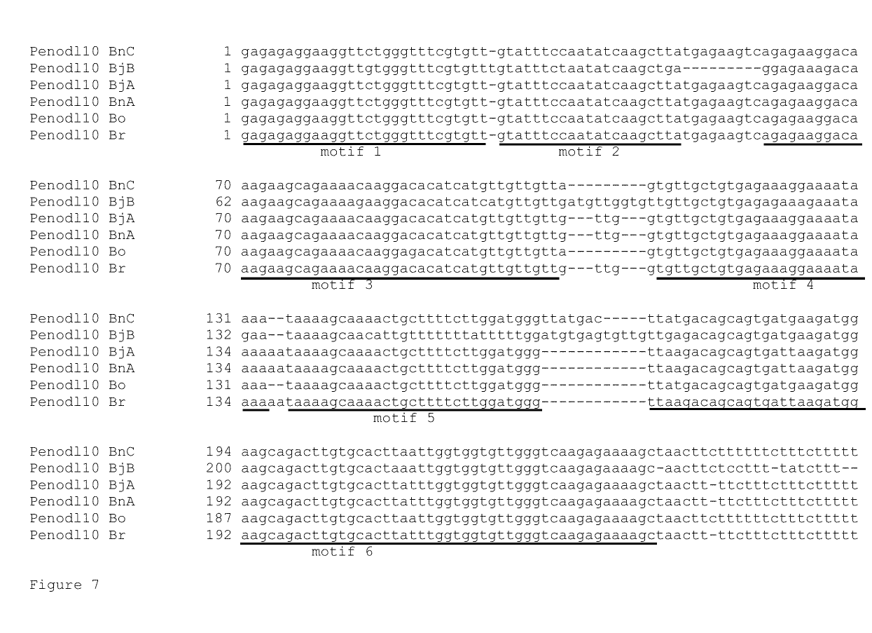

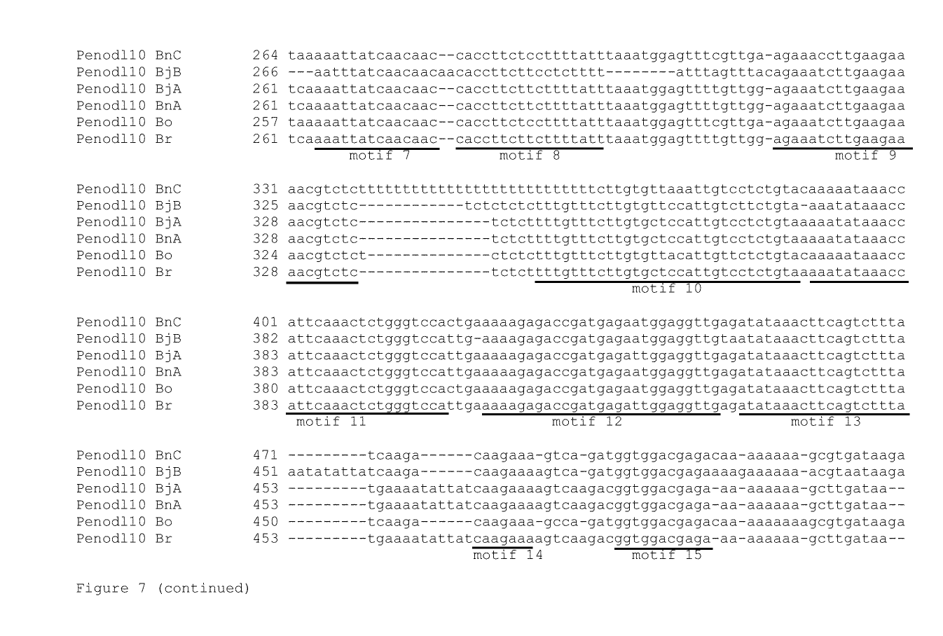

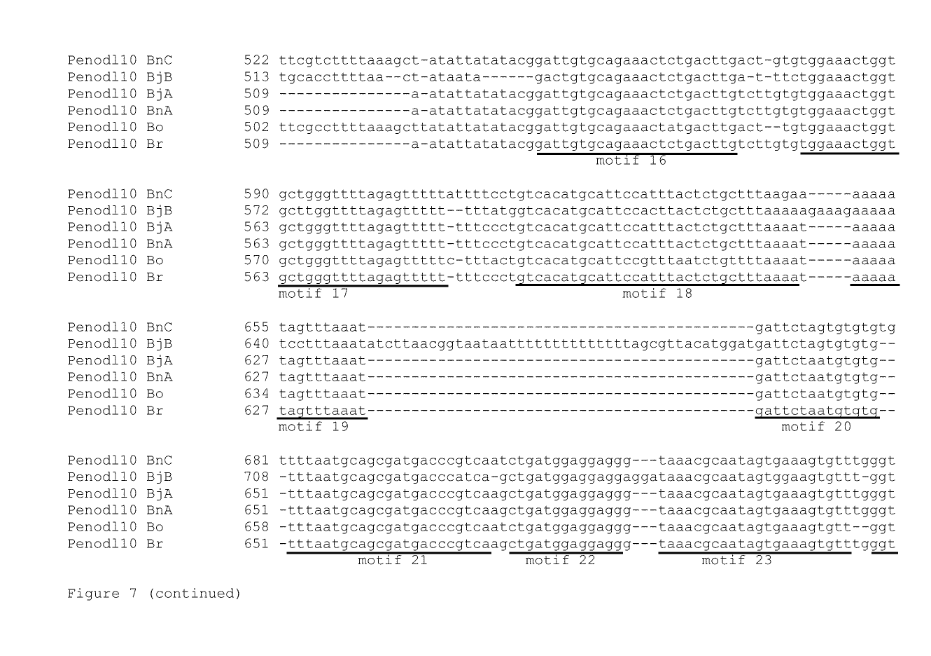

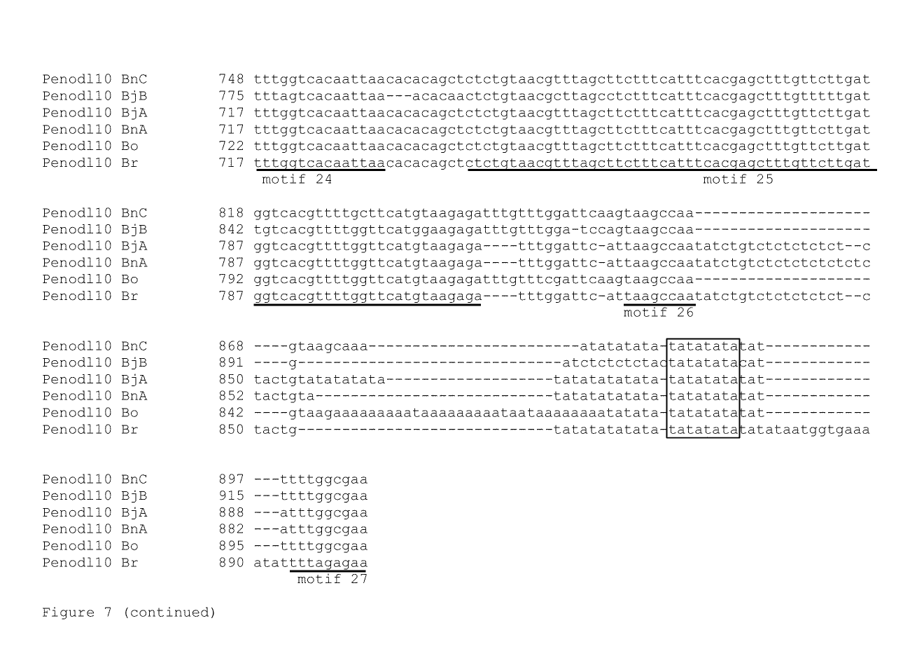

FIG. 7 shows the alignment of the 5′ end sequence of the promoter sequences (SEQ ID NO: 2 to SEQ ID NO: 7). These promoters share a surprisingly high level of conservation in this region. Twenty five consensus sequences (motifs) were identified. The promoters described herein each comprise the following motifs in their sequence:
a. motif 1 is given in SEQ ID NO: 20;
b. motif 2 is given in SEQ ID NO: 21;
c. motif 3 is given in SEQ ID NO: 22;
d. motif 4 is given in SEQ ID NO: 23;
e. motif 5 is given in SEQ ID NO: 24;
f. motif 6 is given in SEQ ID NO: 25;
g. motif 7 is given in SEQ ID NO: 26;
h. motif 8 is given in SEQ ID NO: 27;
i. motif 9 is given in SEQ ID NO: 28;
j. motif 10 is given in SEQ ID NO: 29;
k. motif 11 is given in SEQ ID NO: 30;
l. motif 12 is given in SEQ ID NO: 31;
m. motif 13 is given in SEQ ID NO: 32;

1n. motif 14 has the sequence caagaaa;
o. motif 15 is given in SEQ ID NO: 33;
p. motif 16 is given in SEQ ID NO: 34;
q. motif 17 is given in SEQ ID NO: 35;
r. motif 18 is given in SEQ ID NO: 36;
s. motif 19 is given in SEQ ID NO: 37;
t. motif 20 is given in SEQ ID NO: 38;
u. motif 21 is given in SEQ ID NO: 39;
v. motif 22 is given in SEQ ID NO: 40;
w. motif 23 is given in SEQ ID NO: 41;
x. motif 24 is given in SEQ ID NO: 42;
y. motif 25 is given in SEQ ID NO: 43;
z. motif 26 has the sequence taagccaa;
aa. motif 27 has the sequence tttrgmgaa.

The high degree of conservation of these motifs in all analyzed promoter sequences described herein indicate that these motifs are required for the observed seed-specific and endosperm-preferential expression pattern.

Consequently, as Penodl 10 BjB sequence comprises the motifs 1 to 27, it can be concluded that it has endosperm-preferential promoter activity. As Penodl 10 BjA sequence comprises the motifs 1 to 27, it can be concluded that it has endosperm-preferential promoter activity.

More generally, these results indicate that a Brassica promoter comprising the motifs 1 to 27 would have early stage seed-specific and endosperm-preferential promoter activity.

1-2. (canceled)

3. A recombinant gene comprising a nucleic acid having early stage seed-specific and endosperm preferential promoter activity comprising:
a. a nucleic acid comprising a nucleotide sequence of any one of SEQ ID NO: 2 to SEQ ID NO: 7 or a functional fragment thereof; or
b. a nucleic acid comprising a nucleotide sequence having at least 80% sequence identity to any one of SEQ ID NO: 2 to SEQ ID NO: 7, or a functional fragment thereof,
operably linked to a heterologous nucleic acid sequence encoding an expression product of interest, and optionally a transcription termination and polyadenylation sequence.

4. The recombinant gene according to claim 3, wherein the expression product of interest is an RNA molecule capable of modulating the expression of a gene or is a protein.

5. A host cell comprising the recombinant gene according to claim 3.

6. The host cell of claim 5, which is an E. coli cell, an Agrobacterium cell, a yeast cell, an algal cell, or a plant cell.

7. A plant comprising the recombinant gene of claim 3.

8. A plant comprising at least two recombinant genes according to claim 3, wherein said nucleic acid having early stage seed-specific and endosperm preferential promoter activity, and, optionally, the heterologous nucleic acid sequence, is different in each recombinant gene.

9. Seeds obtainable from the plant according to claim 7.

10. (canceled)

11. A method of producing a transgenic plant comprising:
a. introducing or providing the recombinant gene according to claim 3 to a plant cell to create transgenic cells; and
b. regenerating transgenic plants from said transgenic cell.

12. A method of effecting early stage seed-specific and endosperm preferential expression of a nucleic acid comprising introducing the recombinant gene according to claim 3 into the genome of a plant.

13. A method for altering seed properties of a plant or to produce a commercially relevant product in a plant, comprising introducing the recombinant gene according to claim 3 into the genome of a plant.

14-16. (canceled)

17. The method according to claim 11, wherein said plant is a seed crop plant.

18. A method of producing food, feed, or an industrial product comprising
preparing food, feed or industrial product from the plant of claim 7 or part thereof.

19. The method of claim 18, wherein
a) the food or feed is oil, meal, grain, starch, flour or protein; or
b) the industrial product is biofuel, fiber, industrial chemicals, a pharmaceutical or a nutraceutical.

20. The recombinant gene according to claim 3, wherein said nucleic acid having early stage seed-specific and endosperm preferential promoter activity comprises
a. the nucleotide sequence of SEQ ID NO: 20;
b. the nucleotide sequence of SEQ ID NO: 21;
c. the nucleotide sequence of SEQ ID NO: 22;
d. the nucleotide sequence of SEQ ID NO: 23;
e. the nucleotide sequence of SEQ ID NO: 24;
f. the nucleotide sequence of SEQ ID NO: 25;
g. the nucleotide sequence of SEQ ID NO: 26;
h. the nucleotide sequence of SEQ ID NO: 27;
i. the nucleotide sequence of SEQ ID NO: 28;
j. the nucleotide sequence of SEQ ID NO: 29;
k. the nucleotide sequence of SEQ ID NO: 30;
l. the nucleotide sequence of SEQ ID NO: 31;
m. the nucleotide sequence of SEQ ID NO: 32;
n. the nucleotide sequence caagaaa;
o. the nucleotide sequence of SEQ ID NO: 33;
p. the nucleotide sequence of SEQ ID NO: 34;
q. the nucleotide sequence of SEQ ID NO: 35;
r. the nucleotide sequence of SEQ ID NO: 36;
s. the nucleotide sequence of SEQ ID NO: 37;
t. the nucleotide sequence of SEQ ID NO: 38;
u. the nucleotide sequence of SEQ ID NO: 39;
v. the nucleotide sequence of SEQ ID NO: 40;
w. the nucleotide sequence of SEQ ID NO: 41;
x. the nucleotide sequence of SEQ ID NO: 42;
y. the nucleotide sequence of SEQ ID NO: 43;
z. the nucleotide sequence taagccaa; and
aa. the nucleotide sequence tttrgmgaa.

21. The recombinant gene according to claim 3, operably linked to a transcription termination and polyadenylation region functional in plants.

22. A method of effecting early stage seed-specific and endosperm preferential expression of a nucleic acid comprising providing the plant according to claim 7.

23. A method for altering seed properties of a plant or to produce a commercially relevant product in a plant, comprising providing the plant according to claim 7.


SEQUENCE LISTING (43 sequences)

SEQ ID NO: 1 - DNA, 6140 bp, Artificial Sequence
  nucleotide sequence of the T-DNA Penodl10::GUS
aattacaacggtatatatcctgccagtactttgtggcgctctatcatagctataaacctattcagcacaatatcgattaagggccccctcgagggcgatcgctacgtacctgcaggcccgggttaattaagcggccgcgagagaggaaggttctgggtttcgtgttgtatttccaatatcaagcttatgagaagtcagagaaggacaaagaagcagaaaacaaggacacatcatgttgttgttagtgttgctgtgagaaaggaaaataaaataaaagcaaaactgcttttcttggatgggttatgacttatgacagcagtgatgaagatggaagcagacttgtgcacttaattggtggtgttgggtcaagagaaaagctaacttcttttttctttcttttttaaaaattatcaacaaccaccttctccttttatttaaatggagtttcgttgaagaaaccttgaagaaaacgtctcttttttttttttttttttttttttttcttgtgttaaattgtcctctgtacaaaaataaaccattcaaactctgggtccactgaaaaagagaccgatgagaatggaggttgagatataaacttcagtctttatcaagacaagaaagtcagatggtggacgagacaaaaaaaagcgtgataagattcgtcttttaaagctatattatatacggattgtgcagaaactctgacttgactgtgtggaaactggtgctgggttttagagtttttattttcctgtcacatgcattccatttactctgctttaagaaaaaaatagtttaaatgattctagtgtgtgtgttttaatgcagcgatgacccgtcaatctgatggaggagggtaaacgcaatagtgaaagtgtttgggttttggtcacaattaacacacagctctctgtaacgtttagcttctttcatttcacgagctttgttcttgatggtcacgttttgcttcatgtaagagatttgtttggattcaagtaagccaagtaagcaaaatatatatatatatatatatttttggcgaatccacaatacacacaaaaaacatcatcaaaaactcaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggcgcgtggcgcgcctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgaattcgatatcattaccctgttatccctaaagcttattaatataacttcgtatagcatacattatacgaagttatgtttcaaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacccgtctaggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagataaacataacttcgtatagcatacattatacgaagttatcaaaacgtcgtgagacagtttggttaactataacggtcctaaggtagcgatcgaggcattacggcattacggcactcgcgagggtccgaatctatgtcgggtgcggagaaagaggtaatgaaatggcaattcgagcatggagccatttacaattgaatatatcctgccg

SEQ ID NO: 2 - DNA, 943 bp, Brassica napus
gagagaggaaggttctgggtttcgtgttgtatttccaatatcaagcttatgagaagtcagagaaggacaaagaagcagaaaacaaggacacatcatgttgttgttagtgttgctgtgagaaaggaaaataaaataaaagcaaaactgcttttcttggatgggttatgacttatgacagcagtgatgaagatggaagcagacttgtgcacttaattggtggtgttgggtcaagagaaaagctaacttcttttttctttcttttttaaaaattatcaacaaccaccttctccttttatttaaatggagtttcgttgaagaaaccttgaagaaaacgtctctttttttttttttttttttttttttttcttgtgttaaattgtcctctgtacaaaaataaaccattcaaactctgggtccactgaaaaagagaccgatgagaatggaggttgagatataaacttcagtctttatcaagacaagaaagtcagatggtggacgagacaaaaaaaagcgtgataagattcgtcttttaaagctatattatatacggattgtgcagaaactctgacttgactgtgtggaaactggtgctgggttttagagtttttattttcctgtcacatgcattccatttactctgctttaagaaaaaaatagtttaaatgattctagtgtgtgtgttttaatgcagcgatgacccgtcaatctgatggaggagggtaaacgcaatagtgaaagtgtttgggttttggtcacaattaacacacagctctctgtaacgtttagcttctttcatttcacgagctttgttcttgatggtcacgttttgcttcatgtaagagatttgtttggattcaagtaagccaagtaagcaaaatatatatatatatatatatttttggcgaatccacaatacacacaaaaaacatcatcaaaaactcac

SEQ ID NO: 3 - DNA, 929 bp, Brassica napus
gagagaggaaggttctgggtttcgtgttgtatttccaatatcaagcttatgagaagtcagagaaggacaaagaagcagaaaacaaggacacatcatgttgttgttgttggtgttgctgtgagaaaggaaaataaaaaataaaagcaaaactgcttttcttggatgggttaagacagcagtgattaagatggaagcagacttgtgcacttatttggtggtgttgggtcaagagaaaagctaacttttctttctttcttttttcaaaattatcaacaaccaccttcttcttttatttaaatggagttttgttggagaaatcttgaagaaaacgtctctctcttttgtttcttgtgctccattgtcctctgtaaaaatataaaccattcaaactctgggtccattgaaaaagagaccgatgagaatggaggttgagatataaacttcagtctttatgaaaatattatcaagaaaagtcaagacggtggacgagaaaaaaaaagcttgataaaatattatatacggattgtgcagaaactctgacttgtcttgtgtggaaactggtgctgggttttagagttttttttccctgtcacatgcattccatttactctgctttaaaataaaaatagtttaaatgattctaatgtgtgtttaatgcagcgatgacccgtcaagctgatggaggagggtaaacgcaatagtgaaagtgtttgggttttggtcacaattaacacacagctctctgtaacgtttagcttctttcatttcacgagctttgttcttgatggtcacgttttggttcatgtaagagatttggattcattaagccaatatctgtctctctctctctctactgtatatatatatatatatatatatatatttggcgaattcacaaaacacacaaaaaacatcatcaaaaactcaca

SEQ ID NO: 4 - DNA, 943 bp, Brassica oleracea
gagagaggaaggttctgggtttcgtgttgtatttccaatatcaagcttatgagaagtcagagaaggacaaagaagcagaaaacaaggagacatcatgttgttgttagtgttgctgtgagaaaggaaaataaaataaaagcaaaactgcttttcttggatgggttatgacagcagtgatgaagatggaagcagacttgtgcacttaattggtggtgttgggtcaagagaaaagctaacttcttttttctttcttttttaaaaattatcaacaaccaccttctccttttatttaaatggagtttcgttgaagaaatcttgaagaaaacgtctctctctctttgtttcttgtgttacattgttctctgtacaaaaataaaccattcaaactctgggtccactgaaaaagagaccgatgagaatggaggttgagatataaacttcagtctttatcaagacaagaaagccagatggtggacgagacaaaaaaaaagcgtgataagattcgccttttaaagcttatattatatacggattgtgcagaaactatgacttgacttgtggaaactggtgctgggttttagagtttttctttactgtcacatgcattccgtttaatctgttttaaaataaaaatagtttaaatgattctaatgtgtgtttaatgcagcgatgacccgtcaatctgatggaggagggtaaacgcaatagtgaaagtgttggttttggtcacaattaacacacagctctctgtaacgtttagcttctttcatttcacgagctttgttcttgatggtcacgttttggttcatgtaagagatttgtttcgattcaagtaagccaagtaagaaaaaaaaataaaaaaaaataataaaaaaaatatatatatatatatatttttggcgaattcacaatacacacaaaaaaacatcatcaaaaactcaca

SEQ ID NO: 5 - DNA, 1720 bp, Brassica rapa
gagagaggaaggttctgggtttcgtgttgtatttccaatatcaagcttatgagaagtcagagaaggacaaagaagcagaaaacaaggacacatcatgttgttgttgttggtgttgctgtgagaaaggaaaataaaaaataaaagcaaaactgcttttcttggatgggttaagacagcagtgattaagatggaagcagacttgtgcacttatttggtggtgttgggtcaagagaaaagctaacttttctttctttcttttttcaaaattatcaacaaccaccttcttcttttatttaaatggagttttgttggagaaatcttgaagaaaacgtctctctcttttgtttcttgtgctccattgtcctctgtaaaaatataaaccattcaaactctgggtccattgaaaaagagaccgatgagattggaggttgagatataaacttcagtctttatgaaaatattatcaagaaaagtcaagacggtggacgagaaaaaaaaagcttgataaaatattatatacggattgtgcagaaactctgacttgtcttgtgtggaaactggtgctgggttttagagttttttttccctgtcacatgcattccatttactctgctttaaaataaaaatagtttaaatgattctaatgtgtgtttaatgcagcgatgacccgtcaagctgatggaggagggtaaacgcaatagtgaaagtgtttgggttttggtcacaattaacacacagctctctgtaacgtttagcttctttcatttcacgagctttgttcttgatggtcacgttttggttcatgtaagagatttggattcattaagccaatatctgtctctctctctctactgtatatatatatatatatatatatataatggtgaaaatattttagagaaaaacttcatatgaaacaaaggtaaacgtatcaagtcagatgtctgtactcaaacttcaaaattttctttcggggagatcgaatttagcttattacttttaagctcagttaaacattttgctcgacgtcttttttttttatcagcccataatatagaactagtgggcagtggtttcaaaactggaggtatgaacttaagcaaaatatataaatcatgtttaccaaaaaaaataaaagaaattattgtaccaaattgattaggacaatacatgtatcagaaatgtaagttacacaccgacgaatggtgatacatagctttcagaattatgctctggcaaggcactaattttaaaggatttgtattgaggaaacgggagacataagagtcgcttgtttcacaagaaagaaaataaaatatccattatttcaaacataggattactctcatgaacacactattggttgtttcttggttagcaagcagacaaaaaaaagccagttgagtcgaaaagcgagcatccattaaaattccgtgaaagagacaggtcggaaatagtttttggaaaacaaagaaacgtctgttatagatgggaaataaattatcgtgaacttgataattgataagtgataatgcattattatatgcatcagtaaacatgtataagttgattgaagaactgataattgttattttaaacgacaagaaagcttgggccggaatgattggtagattgtgattcacttattgttgggttccaccaagataattacctcatgttgtggacctctagagtatgtttgggtatcaggtgtctcaca

SEQ ID NO: 6 - DNA, 978 bp, Brassica juncea
gagagaggaaggttgtgggtttcgtgtttgtatttctaatatcaagctgaggagaaagacaaagaagcagaaaagaaggacacatcatcatgttgttgatgttggtgttgttgctgtgagagaaagaaatagaataaaagcaacattgtttttttatttttggatgtgagtgttgttgagacagcagtgatgaagatggaagcagacttgtgcactaaattggtggtgttgggtcaagagaaaagcaacttctccttttatctttaatttatcaacaacaacaccttcttcctcttttatttagtttacagaaatcttgaagaaaacgtctctctctctctttgtttcttgtgttccattgtcttctgtaaaatataaaccattcaaactctgggtccattgaaaagagaccgatgagaatggaggttgtaatataaacttcagtctttaaatatattatcaagacaagaaaagtcagatggtggacgagaaaagaaaaaaacgtaataagatgcaccttttaactataatagactgtgcagaaactctgacttgatttctggaaactggtgcttggttttagagttttttttatggtcacatgcattccacttactctgctttaaaaagaaagaaaaatcctttaaatatcttaacggtaataattttttttttttttagcgttacatggatgattctagtgtgtgtttaatgcagcgatgacccatcagctgatggaggaggaggataaacgcaatagtggaagtgtttggttttagtcacaattaaacacaactctgtaacgcttagcctctttcatttcacgagctttgtttttgattgtcacgttttggttcatggaagagatttgtttggatccagtaagccaagatctctctctactatatatacatttttggcgaattcaccaaacacacaaaaaacatcatcaatatcacaatgtcttcttcgcttatg

SEQ ID NO: 7 - DNA, 935 bp, Brassica juncea
gagagaggaaggttctgggtttcgtgttgtatttccaatatcaagcttatgagaagtcagagaaggacaaagaagcagaaaacaaggacacatcatgttgttgttgttggtgttgctgtgagaaaggaaaataaaaaataaaagcaaaactgcttttcttggatgggttaagacagcagtgattaagatggaagcagacttgtgcacttatttggtggtgttgggtcaagagaaaagctaacttttctttctttcttttttcaaaattatcaacaaccaccttcttcttttatttaaatggagttttgttggagaaatcttgaagaaaacgtctctctcttttgtttcttgtgctccattgtcctctgtaaaaatataaaccattcaaactctgggtccattgaaaaagagaccgatgagattggaggttgagatataaacttcagtctttatgaaaatattatcaagaaaagtcaagacggtggacgagaaaaaaaaagcttgataaaatattatatacggattgtgcagaaactctgacttgtcttgtgtggaaactggtgctgggttttagagttttttttccctgtcacatgcattccatttactctgctttaaaataaaaatagtttaaatgattctaatgtgtgtttaatgcagcgatgacccgtcaagctgatggaggagggtaaacgcaatagtgaaagtgtttgggttttggtcacaattaacacacagctctctgtaacgtttagcttctttcatttcacgagctttgttcttgatggtcacgttttggttcatgtaagagatttggattcattaagccaatatctgtctctctctctctactgtatatatatatatatatatatatatatatatatatttggcgaattcacaaaacacacaaaaaacatcatcaaaaactcaca

SEQ ID NO: 8 - protein, 168 aa, Brassica napus
MTRQSDGGGEILVGGKSNTWKVPESTEETLNQWSERTRFKIGDALLWKYNAENDSVMQVSKKDYERCDRSEPIKGYKDGHTKIELKRSGPFYFISGEDGHCQRGEKLLVVVLSPNHNRSYTADAPLTTPVTDKGSDHVLINQAQRSIATACLIFLLPLVLFGLVLLPL

SEQ ID NO: 9 - protein, 173 aa, Brassica napus
MSSLMICYLFLLLSLLCEGREILVGGKSNTWKVPESTEETLNQWSERTRFKIGDSLLWKYNAENDSVMQVSQKDYERCDRSEPIRGYKDGHTKIELKRSGPFYFISGEEGYCQRGEKLLVVVLSPNHNRSYAADAPLTAPVTKNKGSDHVLINQAQRSIATVCFLVLPLVLLV

SEQ ID NO: 10 - protein, 179 aa, Brassica oleracea
MSSLMICYLFLLLCLLCEGREILVGGKSNTWKVPESTEETLNQWSERTRFKIGDALLWKYNAENDSVMQVSKKDYERCDRLEPIKGYKDGHTKIELKRSGPFYFISGEDGHCQRGEKLLVVVLSPNHNRSYTADAPLTTPVTDKGSDHVLINQAQRSIATACLIFLLPLVLFGLVLLPL

SEQ ID NO: 11 - protein, 173 aa, Brassica rapa; identical to SEQ ID NO: 9

SEQ ID NO: 12 - protein, 168 aa, Brassica juncea
MCCLFLLLCLLCEGREILVGGKSNTWKVPDSTEETLNQWSERTRFKIGDSLLWKYNAENDSVMQVSQKDYERCDRSEPIRGYKDGHTKIELNRSGPFYFISGEEGRCQRGEKLLVVVLSPNHNRSYAADAPLTAPVTKNKGSDHVLINQAQRSIATVCFLVLPLVLLV

SEQ ID NO: 13 - protein, 173 aa, Brassica juncea; identical to SEQ ID NO: 9

SEQ ID NO: 14 - DNA, 507 bp, Brassica napus
atgacccgtcaatctgatggaggaggggagattctagtgggaggcaagtccaacacatggaaagttccagagtctacagaagaaacactaaaccaatggtcagagagaacacgattcaaaattggagatgctctcctgtggaaatacaatgcggagaatgactcagtgatgcaagtgagtaagaaagattacgagagatgcgacagatcagagcctataaaaggatacaaagatggtcacacgaagatagagctaaagagatcaggtccgttttacttcatcagtggcgaagatgggcattgccaaagaggagagaagcttcttgttgttgtcttgtctccaaatcacaacagaagctatacagctgatgctcctttaaccacaccggtcacagacaagggaagtgatcatgttctgatcaatcaagcacaaagatccattgcaactgcttgtttaatttttcttcttccattagtcctttttggtttagtcttgttacctctttga

SEQ ID NO: 15 - DNA, 522 bp, Brassica napus
atgtcttcgcttatgatatgttatttgtttctgttattgtctttgttgtgtgaaggcagggagattctagtgggaggcaagtccaacacatggaaagttccagagtctacagaagaaacactaaaccaatggtcagagaggacaagattcaaaattggagactctctcttgtggaaatacaatgcggagaatgactcagtgatgcaagtgagtcaaaaagactacgagagatgcgacagatcagaacctataagaggatacaaagatggtcacacaaagatagagctaaagagatcaggtccgttttacttcatcagtggcgaagaagggtattgccaaagaggagagaagcttcttgttgttgtcttgtctccaaatcacaacagaagctatgcagctgatgctcctttaaccgctccggtcacaaaaaacaagggaagtgatcatgttctgatcaaccaagcacaaagatccattgcaactgtttgtttccttgttcttccattagtccttttggtttag

SEQ ID NO: 16 - DNA, 540 bp, Brassica oleracea
atgtcttcgcttatgatatgttatttgtttctgttgttatgtttgttatgtgaaggcagggagattctagtgggaggcaagtccaacacatggaaagttccagagtctacagaagaaacactaaaccaatggtcagagagaacacgattcaaaattggagatgctctcctgtggaaatacaatgcggagaatgactcagtgatgcaagtgagtaagaaagattacgagagatgcgacagattagagcctataaaaggatacaaagatggtcacacgaagatagagctaaagagatcaggtccgttttacttcatcagtggcgaagatgggcattgccaaagaggggagaagcttcttgttgttgtcttgtctccaaatcacaacagaagctatacagctgatgctcctttaaccacaccggtcacagacaagggaagtgatcatgttctgatcaatcaagcacaaagatccattgcaactgcttgtttaatttttcttcttccattagtcctttttggtttagtcttgttacctctttga

SEQ ID NO: 17 - DNA, 522 bp, Brassica rapa; identical to SEQ ID NO: 15

SEQ ID NO: 18 - DNA, 507 bp, Brassica juncea
atgtgttgtttgtttctgttattgtgtttgttatgtgaaggcagggagattctagtgggaggcaagtctaacacatggaaggttccagattctacagaagaaacactaaaccaatggtcagagagaacaagattcaaaattggagattctctcttgtggaaatacaatgcggagaatgactcagtgatgcaagtgagtcaaaaagattacgagagatgcgacagatcagagcctataagagggtacaaagatggtcacaccaagatagagctaaacagatcaggtcctttttacttcatcagtggtgaagaaggtcgttgtcagagaggagagaagcttcttgttgtggtcttgtctccaaatcacaacagaagctatgcagctgatgctcctttaaccgctccggtcacaaaaaacaagggaagtgatcatgttctgatcaaccaagcacaaagatccattgcaactgtttgtttccttgttcttccattagtccttttggtttag

SEQ ID NO: 19 - DNA, 522 bp, Brassica juncea; identical to SEQ ID NO: 15

SEQ ID NO: 20 - DNA, 28 bp, Artificial Sequence
  motif 1
gagagaggaaggttstgggtttcgtgtt

SEQ ID NO: 21 - DNA, 21 bp, Artificial Sequence
  motif 2
gtatttcyaatatcaagctka

SEQ ID NO: 22 - DNA, 47 bp, Artificial Sequence
  motif 3
rgagaargacaaagaagcagaaaasaaggasacatcatswtgttgtt

SEQ ID NO: 23 - DNA, 26 bp, Artificial Sequence
  motif 4
tgttgctgtgagararrraaataraa

SEQ ID NO: 24 - DNA, 29 bp, Artificial Sequence
  motif 5
taaaagcaamaytgyttttytwkkwtkgg

SEQ ID NO: 25 - DNA, 71 bp, Artificial Sequence
  motif 6
ttrwgacagcagtgatkaagatggaagcagacttgtgcactwawttggtggtgttgggtcaagagaaaagc

SEQ ID NO: 26 - DNA, 14 bp, Artificial Sequence
  motif 7
aawttatcaacaac

SEQ ID NO: 27 - DNA, 17 bp, Artificial Sequence
  motif 8
caccttctycytytwtt

SEQ ID NO: 28 - DNA, 23 bp, Artificial Sequence
  motif 9
agaaaycttgaagaaaacgtctc

SEQ ID NO: 29 - DNA, 30 bp, Artificial Sequence
  motif 10
tttktttcttgtgytmmattgtyytctgta

SEQ ID NO: 30 - DNA, 29 bp, Artificial Sequence
  motif 11
aaawataaaccattcaaactctgggtcca

SEQ ID NO: 31 - DNA, 27 bp, Artificial Sequence
  motif 12
aaaagagaccgatgagawtggaggttg

SEQ ID NO: 32 - DNA, 19 bp, Artificial Sequence
  motif 13
atataaacttcagtcttta

SEQ ID NO: 33 - DNA, 11 bp, Artificial Sequence
  motif 15
ggtggacgaga

SEQ ID NO: 34 - DNA, 23 bp, Artificial Sequence
  motif 16
gaytgtgcagaaactmtgacttg

SEQ ID NO: 35 - DNA, 30 bp, Artificial Sequence
  motif 17
tggaaactggtgctkggttttagagttttt

SEQ ID NO: 36 - DNA, 32 bp, Artificial Sequence
  motif 18
gtcacatgcattccryttamtctgytttaara

SEQ ID NO: 37 - DNA, 15 bp, Artificial Sequence
  motif 19
aaaaatmstttaaat

SEQ ID NO: 38 - DNA, 14 bp, Artificial Sequence
  motif 20
gattctartgtgtg

SEQ ID NO: 39 - DNA, 23 bp, Artificial Sequence
  motif 21
tttaatgcagcgatgacccrtca

SEQ ID NO: 40 - DNA, 13 bp, Artificial Sequence
  motif 22
ctgatggaggagg

SEQ ID NO: 41 - DNA, 22 bp, Artificial Sequence
  motif 23
taaacgcaatagtgraagtgtt

SEQ ID NO: 42 - DNA, 18 bp, Artificial Sequence
  motif 24
ggttttrgtcacaattaa

SEQ ID NO: 43 - DNA, 72 bp, Artificial Sequence
  motif 25
ctctgtaacgyttagcytctttcatttcacgagctttgttyttgatkgtcacgttttgsttcatgkaagaga